(19) United States
(12) Patent Application Publication
(10) Pub. No.: US 2025/0385009 A1
(43) Pub. Date: Dec. 18, 2025

(54) SYSTEM AND METHOD FOR GENERATING A RISK SCORE

(71) Applicant: ORPYX MEDICAL TECHNOLOGIES INC., CALGARY (CA)

(72) Inventors: EMILY T. BRAY, SAN FRANCISCO, CA (US); COURTNEY BLAINE BACHUS, CALGARY (CA); EVAN PATRICK MINTY, CALGARY (CA); KAREN MERLET SMITH, LAKE VILLA, IL (US); JULIA BREANNE EVERETT, CALGARY (CA); BROCK ARMSTRONG LIDEN, PICKERINGTON, OH (US)

(73) Assignee: ORPYX MEDICAL TECHNOLOGIES INC., CALGARY, AB (CA)

(21) Appl. No.: 18/740,647

(22) Filed: Jun. 12, 2024

(51) Int. Cl. G16H 50/30; A61B 5/00; A61B 5/01; G16H 10/60

(52) U.S. Cl. CPC G16H 50/30; A61B 5/01; A61B 5/6807; A61B 5/7275; A61B 5/746; A61B 5/7475; G16H 10/60

(57) ABSTRACT

A method for generating a risk score includes: for each user in a plurality of users, collecting a plurality of sensor readings over a time period using an input device worn by the user, the input device configured to collect the plurality of sensor readings; calculating one or more comparison metrics based on the plurality of sensor readings; determining whether the one or more comparison metrics satisfies one or more threshold requirements; generating a plurality of information alerts if the one or more comparison metrics does not satisfy the one or more threshold requirements in the time period, each information alert corresponding to an information alert type; generating a risk factor for each information alert based on the information alert type; generating one or more alert totals for the time period based on the plurality of information alerts; generating a risk score for the user based on each of the risk factors and the one or more alert totals; and ordering the plurality of users in a list according to the risk score for each user.

### FIELD

This document relates to preventing the development of diabetic foot ulcers. More specifically, this document relates to systems and methods for generating a risk score for a user from a plurality of sensors disposed in an input device, the risk score indicating the user's risk of developing a diabetic foot ulcer.

### BACKGROUND

US Patent Application Publication No. 20210319887A1 (Derrick, Jr. et al.) discloses a method for treating diabetes based on social determinants of health. The method includes analyzing social determinants of health and generating a modifiable social determinants of health (mSDOH)-informed patient risk score. The mSDOH-informed patient risk score may be used to evaluate compliance of the patient with their care plan and to alert the patient or a healthcare team to the patient's likelihood of cessation from the care plan.

### SUMMARY

The following summary is intended to introduce the reader to various aspects of the detailed description, but not to define or delimit any invention.

A method for vascular assessment alert generation is disclosed. According to some aspects, a method for vascular assessment alert generation includes collecting a plurality of temperature readings over a time period by an input device, the time period including a first time and a second time, the input device worn by the user and configured to collect the plurality of temperature readings; determining a first temperature difference for the first time by comparing a first temperature reading at a first plantar location to a first temperature reading at a second plantar location; determining a second temperature difference for the second time by comparing a second temperature reading at the first plantar location to a second temperature reading at the second plantar location; and generating a vascular assessment requirement alert if the first temperature difference and the second temperature difference exceed a temperature threshold.

In some examples, the first plantar location or the second plantar location is a location underfoot of the user.

In some examples, the first plantar location and the second plantar location are on a same foot of the user.

In some examples, the first temperature reading at the second plantar location is an average of the plurality of temperature readings at all plantar locations collected by the input device at the first time, and the second temperature reading at the second plantar location is an average of the plurality of temperature readings at all plantar locations collected by the input device for the second time.

In some examples, the first plantar location is on a first foot of the user and the second plantar location is on a second foot of the user.

In some examples, the method further includes receiving foot examination data, and the foot examination data indicates a presence of an abnormality on the first foot and/or the second foot of the user.

In some examples, the first time and the second time are consecutive.

In some examples, the second time is at least one hour after the first time. In some examples, the temperature threshold is at least 2.0 degrees Celsius.

In some examples, the method further includes presenting the vascular assessment requirement alert.

In some examples, the vascular assessment requirement alert recommends a vascular assessment including an ankle brachial index test with segmental pressures and a doppler waveform analysis.

In some examples, the method further includes collecting activity data over the time period using the input device, the input device configured to collect the activity data; identifying an occurrence of an activity based on the activity data for the time period; and filtering out changes in the plurality of temperature readings that temporally align with the occurrence of the activity.

In some examples, filtering out the changes in the plurality of temperature readings includes determining rates of change of the plurality of temperature readings for the plurality of temperature readings that temporally align with the occurrence of the activity, and filtering out the plurality of temperature readings when the rates of change exceed a rate of change threshold.

In some examples, the activity is donning the input device, doffing the input device, or a high intensity activity.

In some examples, the activity data includes accelerometer data, gyroscope data, and/or pressure data.

In some examples, the method further includes receiving historical user data of the user; identifying a chronic temperature difference if the historical user data includes a condition that causes chronic limb temperature differences; and normalizing the plurality of temperature readings to eliminate the chronic temperature difference.

In some examples, the condition that causes chronic limb temperature differences is peripheral arterial disease, macrovascular disease, a stroke, an asymmetric neurological disease, muscle mass asymmetry, or edema.

In some examples, the input device is footwear.

In some examples, the input device is an insole or a pair of insoles.

A system for vascular assessment alert generation is disclosed. According to some aspects, a system for vascular assessment alert generation includes an input device worn by a user, the input device including two or more temperature sensors, the two or more temperature sensors configured to collect a plurality of temperature readings from the user over a time period, the time period including a first time and a second time; and a processor in communication with the input device, the processor configured to: receive the plurality of temperature readings from the input device; determine a first temperature difference for the first time by comparing a first temperature reading at a first plantar location to a first temperature reading at a second plantar location; determine a second temperature difference for the second time by comparing a second temperature reading at the first plantar location to a second temperature reading at the second plantar location; and generate a vascular assessment requirement alert if the first temperature difference and the second temperature difference exceed a temperature threshold.

In some examples, the first plantar location or the second plantar location are a location underfoot of the user.

In some examples, the first plantar location and the second plantar location are on a same foot of the user.

In some examples, the first temperature reading at the second plantar location is an average of the plurality of temperature readings collected by the input device at all plantar locations for the first time, and the second temperature reading at the second plantar location is an average of the plurality of temperature readings at all plantar locations for the second time.

In some examples, the first plantar location is on a first foot of the user and the second plantar location is on a second foot of the user.

In some examples, the processor is further configured to receive foot examination data, and the foot examination data indicates a presence of an abnormality on the first foot and/or the second foot of the user.

In some examples, the first time and the second time are consecutive.

In some examples, the second time is at least one hour after the first time. In some examples, the temperature threshold is at least 2.0 degrees Celsius.

In some examples, the system further includes a display, the display configured to present the vascular assessment requirement alert.

In some examples, the vascular assessment requirement alert recommends a vascular assessment including an ankle brachial index test with segmental pressures and a doppler waveform analysis.

In some examples, the input device further includes one or more additional sensors, the one or more additional sensors configured to collect activity data from the user over the time period; and the processor is further configured to: receive the activity data from the input device; identify an occurrence of an activity based on the activity data for the time period; and filter out changes in the plurality of temperature readings that temporally align with the occurrence of the activity.

In some examples, the processor is configured to filter out the changes in the plurality of temperature readings by determining rates of change of the plurality of temperature readings for the plurality of temperature readings that temporally align with the occurrence of the activity, and filtering out the plurality of temperature readings when the rates of change exceed a rate of change threshold.

In some examples, the activity is donning the input device, doffing the input device, or a high intensity activity.

In some examples, the one or more additional sensors includes an accelerometer, a gyroscope, and/or one or more pressure sensors.

In some examples, the processor is further configured to: receive historical user data of the user; identify a chronic temperature difference if the historical user data includes a condition that causes chronic limb temperature differences; and normalize the plurality of temperature readings to eliminate the chronic temperature difference.

In some examples, the condition is peripheral arterial disease, macrovascular disease, a stroke, an asymmetric neurological disease, muscle mass asymmetry, or edema.

In some examples, the input device is footwear.

In some examples, the input device is an insole or a pair of insoles.

A method for generating a step count is disclosed. According to some aspects, a method for generating a step count includes collecting a plurality of accelerometer readings over a time period using an input device, the time period including a previous time period and a current time period, the input device configured to collect the plurality of accelerometer readings and including an insole; collecting a plurality of additional sensor readings over an additional time period using the input device, wherein the current time period includes the additional time period and the input device is configured to collect the plurality of additional sensor readings; determining a rolling step count for the current time period based on the plurality of accelerometer readings; determining a step count for the previous time period based on the plurality of accelerometer readings; and generating a step count goal for a future time period based on the step count for the previous time period, when the rolling step count exceeds a step count threshold and when an additional threshold requirement is not satisfied in the additional time period based on the plurality of additional sensor readings.

In some examples, the method further includes presenting the step count goal for the future time period.

In some examples, the current time period is at least three days.

In some examples, the previous time period or the future time period are at least one month.

In some examples, the previous time period is longer than the current time period.

In some examples, the additional time period is at least one day.

In some examples, the previous time period and the current time period overlap.

In some examples, the step count goal for the future time period is at least 10% higher than the step count for the previous time period.

In some examples, the step count threshold is 50% of a step count baseline.

In some examples, the step count baseline is an average step count of a plurality of previous time periods.

In some examples, collecting a plurality of accelerometer readings over the future time period using the input device; determining a step count for the future time period based on the plurality of accelerometer readings over the future time period; and generating an alert indicating whether the step count for the future time period satisfies the step count goal for the future time period.

In some examples, the plurality of additional sensor readings includes a plurality of pressure readings, the additional threshold requirement is a percentage threshold requirement, and the percentage threshold requirement is not satisfied when a percentage of time spent in an unacceptable pressure state based on the plurality of the pressure readings exceeds a percentage threshold in the additional time period.

In some examples, the percentage of time spent in the unacceptable pressure state is determined using a pressure threshold, and the pressure threshold is 50 mmHg.

In some examples, the percentage threshold is in a range of 40% to 50%.

In some examples, the plurality of additional sensor readings includes a plurality of temperature readings, the additional threshold requirement is a temperature threshold requirement, and the temperature threshold requirement is not satisfied when a first temperature difference and a second temperature difference exceed a temperature threshold.

In some examples, the first temperature difference is determined for a first time in the additional time period by comparing a first temperature reading at a first plantar location to a first temperature reading at a second plantar location; and the second temperature difference is determined for a second time in the additional time period by comparing a second temperature reading at the first plantar location to a second temperature reading at the second plantar location.

In some examples, the temperature threshold is at least 2.0 degrees Celsius.

In some examples, the method further includes generating an additional action alert when the additional threshold requirement is not satisfied in the additional time period; and presenting the additional action alert, and the additional action alert triggers the user to offload a foot pressure.

A system for generating a step count is disclosed. According to some aspects, a system for generating a step count includes an input device worn by a user, the input device including an insole and including: an accelerometer, the accelerometer configured to collect a plurality of accelerometer readings over a time period, wherein the time period includes a previous time period and a current time period; and at least one additional sensor, the at least one additional sensor configured to collect a plurality of additional sensor readings over an additional time period, wherein the current time period includes the additional time period; a processor in communication with the input device, the processor configured to: receive the plurality of accelerometer readings from the input device; receive the plurality of additional sensor readings from the input device; determine a rolling step count for the current time period based on the plurality of accelerometer readings; determine a step count for the previous time period based on the plurality of accelerometer readings; and generate a step count goal for a future time period based on the step count for the previous time period, when the rolling step count exceeds a step count threshold and when an additional threshold requirement is not satisfied in the additional time period based on the plurality of additional sensor readings.

In some examples, the system further includes a display in communication with the processor, and the display is configured to present the step count goal for the future time period.

In some examples, the current time period is at least three days.

In some examples, the previous time period or the future time period are at least one month.

In some examples, the additional time period is at least one day.

In some examples, the previous time period and the current time period overlap.

In some examples, the previous time period is longer than the current time period.

In some examples, the step count goal for the future time period is at least 10% higher than the step count for the previous time period.

In some examples, the step count threshold is 50% of a step count baseline.

In some examples, the step count baseline is an average step count of a plurality of previous time periods.

In some examples, the accelerometer is further configured to collect a plurality of accelerometer readings over the future time period; and the processor is further configured to: receive the plurality of accelerometer readings over the future time period from the input device; determine a step count for the future time period based on the plurality of accelerometer readings over the future time period; and generate an alert indicating whether the step count for the future time period satisfies the step count goal for the future time period.

In some examples, the at least one additional sensor includes one or more pressure sensors, the plurality of additional sensor readings includes a plurality of pressure readings, the additional threshold requirement is a percentage threshold requirement, and the percentage threshold requirement is not satisfied when a percentage of time spent in an unacceptable pressure state based on the plurality of the pressure readings exceeds a percentage threshold in the additional time period.

In some examples, the percentage of time spent in the unacceptable pressure state is determined using a pressure threshold, and the pressure threshold is 50 mmHg.

In some examples, the percentage threshold is in a range of 40% to 50%.

In some examples, the at least one additional sensor includes two or more temperature sensors, the plurality of additional sensor readings includes a plurality of temperature readings, the additional threshold requirement is a temperature threshold requirement, and the temperature threshold requirement is not satisfied when a first temperature difference and a second temperature difference exceed a temperature threshold.

In some examples, the processor is further configured to: receive the plurality of temperature readings from the input device; determine the first temperature difference for a first time in the additional time period by comparing a first temperature reading at a first plantar location to a first temperature reading at a second plantar location; and determine the second temperature difference for a second time in the additional time period by comparing a second temperature reading at the first plantar location to a second temperature reading at the second plantar location.

In some examples, the temperature threshold is at least 2.0 degrees Celsius.

In some examples, the processor is further configured to generating an additional action alert when the additional threshold requirement is not satisfied in the additional time period; and the display is further configured to present the additional action alert, and the additional action alert triggers the user to offload a foot pressure.

A method for generating a risk score is disclosed. In some aspects, a method for generating a risk score includes: for each user in a plurality of users, collecting a plurality of sensor readings over a time period using an input device worn by the user, the input device configured to collect the plurality of sensor readings; calculating one or more comparison metrics based on the plurality of sensor readings; determining whether the one or more comparison metrics satisfies one or more threshold requirements; generating a plurality of information alerts if the one or more comparison metrics does not satisfy the one or more threshold requirements in the time period, each information alert corresponding to an information alert type; generating a risk factor for each information alert based on the information alert type; generating one or more alert totals for the time period based on the plurality of information alerts; generating a risk score for the user based on each of the risk factors and the one or more alert totals; and ordering the plurality of users in a list according to the risk score for each user.

In some examples, ordering the plurality of users in the list includes arranging the plurality of users in descending order according to the risk score for each user.

In some examples, the method further includes generating a care timeline for each user based on the risk score, and high risk scores are associated with urgent care timelines.

In some examples, the information alert type is one of a pressure information alert, a temperature information alert, an adherence information alert, and a combination information alert.

In some examples, the risk factor includes a risk factor qualitative level.

In some examples, the risk factor includes a risk factor quantitative level.

In some examples, generating the one or more alert totals for the time period includes counting a total number of information alerts in the time period.

In some examples, generating the one or more alert totals for the time period includes counting a total number of information alerts corresponding to each information alert type in the time period.

In some examples, the method further includes generating an alert total qualitative level based on the one or more alert totals.

In some examples, the method further includes generating an alert total quantitative level based on the one or more alert totals.

In some examples, the alert total qualitative level or the risk factor qualitative level is a low risk, a medium risk, a medium-high risk, or a high risk.

In some examples, generating the risk score further includes averaging the risk factors and/or the one or more alert totals.

In some examples, generating the risk score further includes calculating a weighted sum of the risk factors and the one or more alert totals.

In some examples, generating the risk score further includes using additional data.

In some examples, the additional data includes foot examination data, and the foot examination data includes a presence or an absence of an abnormality on a foot of the user.

In some examples, the foot examination data further includes location information and measurement information of the abnormality when the presence of the abnormality is detected.

In some examples, the additional data includes sensor alert patterns.

In some examples, the additional data includes scan data for the foot of the user, the scan data obtained using thermography and/or transcutaneous oxygen saturation imaging.

In some examples, the additional data includes historical user data, and the historical user data includes historical ulceration data, historical foot data, historical amputation data, historical foot surgery data, historical social data, nutritional status, historical gait data, historical mobility data, historical medication user, historical comorbidity data, and historical lab test data.

In some examples, the method further includes calculating a tie breaking score when two users have the same risk score.

In some examples, the tie breaking score is calculated using the one or more alert totals.

In some examples, the tie breaking score is calculated using the additional data.

In some examples, the plurality of sensor readings includes a plurality of pressure readings, the one or more comparison metrics includes a percentage of time spent in an unacceptable pressure state based on the plurality of the pressure readings, the threshold requirement includes a percentage threshold requirement, the percentage threshold requirement is not satisfied when the percentage of time spent in the unacceptable pressure state exceeds a percentage threshold, and the plurality of information alerts includes a pressure information alert.

In some examples, the plurality of sensor readings includes a plurality of temperature readings, the one or more comparison metrics includes a first temperature difference and a second temperature difference, the threshold requirement includes a temperature threshold requirement, the temperature threshold requirement is not satisfied when the first temperature difference and the second temperature difference exceed a temperature threshold, and the plurality of information alerts includes a temperature information alert.

In some examples, the one or more comparison metrics includes a usage time, the threshold requirement includes a usage threshold requirement, the usage threshold requirement is not satisfied when the usage time is below a usage threshold, and the plurality of information alerts includes an adherence information alert.

In some examples, the plurality of sensor readings includes a plurality of accelerometer readings, the one or more comparison metrics includes a rolling step count, the threshold requirement includes a step threshold requirement, the step threshold requirement is not satisfied when the rolling step count exceeds a step count threshold, and the plurality of information alerts includes a step count information alert.

In some examples, the plurality of sensor readings includes a plurality of accelerometer readings and a plurality of pressure readings, the one or more comparison metrics includes a rolling step count and a percentage of time spent in an unacceptable pressure state based on the plurality of the pressure readings, the threshold requirement includes a step threshold requirement and a percentage threshold requirement, the step threshold requirement is not satisfied when the rolling step count exceeds a step count threshold and the percentage threshold requirement is not satisfied when the percentage of time spent in the unacceptable pressure state exceeds a percentage threshold, and the plurality of information alerts includes a combination information alert.

In some examples, the plurality of sensor readings includes a plurality of accelerometer readings and a plurality of temperature readings, the one or more comparison metrics includes a rolling step count, a first temperature difference, and a second temperature difference, the threshold requirement includes a step threshold requirement and a temperature threshold requirement, the step threshold requirement is not satisfied when the rolling step count exceeds a step count threshold and the temperature threshold requirement is not satisfied when the first temperature difference and the second temperature difference exceed a temperature threshold, and the plurality of information alerts includes a combination information alert.

In some examples, the input device is footwear.

In some examples, the input device is an insole or a pair of insoles.

A system for generating a risk score is disclosed. In some aspects, a system for generating a risk score includes: an input device worn by each user in a plurality of users, the input device including: a plurality of sensors, the plurality of sensors configured to collect a plurality of sensor readings over a time period; a processor in communication with the input device, the processor configured to: receive the plurality of sensor readings from the input device; calculate one or more comparison metrics based on the plurality of sensor readings; determine whether the one or more comparison metrics satisfies one or more threshold requirements; generate a plurality of information alerts if the one or more comparison metrics does not satisfy the one or more threshold requirements in the time period, each information alert corresponding to an information alert type; generate a risk factor for each information alert based on the information alert type; generate one or more alert totals for the time period based on the plurality of information alerts; generate a risk score for the user based on each of the risk factors and the one or more alert totals; and order the plurality of users in a list according to the risk score for each user.

In some examples, the processor is configured to order the plurality of users in the list in descending order according to the risk score for each user.

In some examples, the processor is further configured to generate a care timeline for each user based on the risk score, and high risk scores are associated with urgent care timelines.

In some examples, the information alert type is one of a pressure information alert, a temperature information alert, an adherence information alert, and a combination information alert.

In some examples, the risk factor includes a risk factor qualitative level.

In some examples, the risk factor includes a risk factor quantitative level.

In some examples, generating the one or more alert totals for the time period includes counting a total number of information alerts in the time period.

In some examples, the processor is configured to generate the one or more alert totals for the time period by counting a total number of information alerts corresponding to each information alert type in the time period.

In some examples, the processor is further configured to generate an alert total qualitative level based on the one or more alert totals.

In some examples, the processor is further configured to generate an alert total quantitative level based on the one or more alert totals.

In some examples, the alert total qualitative level or the risk factor qualitative level is a low risk, a medium risk, a medium-high risk, or a high risk.

In some examples, the processor is configured to generate the risk score by averaging the risk factors and/or the one or more alert totals.

In some examples, the processor is configured to compute the risk score by calculating a weighted sum of the risk factors and the one or more alert totals.

In some examples, the processor is further configured to generate the risk score using additional data.

In some examples, the additional data includes foot examination data, and the foot examination data includes a presence or an absence of an abnormality on a foot of the user.

In some examples, the foot examination data further includes location information and measurement information of the abnormality when the presence of the abnormality is detected.

In some examples, the additional data includes sensor alert patterns.

In some examples, the additional data includes scan data for the foot of the user, the scan data obtained using thermography and/or transcutaneous oxygen saturation imaging.

In some examples, the additional data includes historical user data, and the historical user data includes historical ulceration data, historical foot data, historical amputation data, historical foot surgery data, historical social data, nutritional status, historical gait data, historical mobility data, historical medication user, historical comorbidity data, and historical lab test data.

In some examples, the processor is further configured to calculate a tie breaking score when two users have the same risk score.

In some examples, the processor is configured to calculate the tie breaking score using the one or more alert totals.

In some examples, the tie breaking score is calculated using the additional data.

In some examples, the plurality of sensor readings includes a plurality of pressure readings, the one or more comparison metrics includes a percentage of time spent in an unacceptable pressure state based on the plurality of the pressure readings, the threshold requirement includes a percentage threshold requirement, the percentage threshold requirement is not satisfied when the percentage of time spent in the unacceptable pressure state exceeds a percentage threshold, and the plurality of information alerts includes a pressure information alert.

In some examples, the plurality of sensor readings includes a plurality of temperature readings, the one or more comparison metrics includes a first temperature difference and a second temperature difference, the threshold requirement includes a temperature threshold requirement, the temperature threshold requirement is not satisfied when the first temperature difference and the second temperature difference exceed a temperature threshold, and the plurality of information alerts includes a temperature information alert.

In some examples, the one or more comparison metrics includes a usage time, the threshold requirement includes a usage threshold requirement, the usage threshold requirement is not satisfied when the usage time is below a usage threshold, and the plurality of information alerts includes an adherence information alert.

In some examples, the plurality of sensor readings includes a plurality of accelerometer readings, the one or more comparison metrics includes a rolling step count, the threshold requirement includes a step threshold requirement, the step threshold requirement is not satisfied when the rolling step count exceeds a step count threshold, and the plurality of information alerts includes a step count information alert.

In some examples, the plurality of sensor readings includes a plurality of accelerometer readings and a plurality of pressure readings, the one or more comparison metrics includes a rolling step count and a percentage of time spent in an unacceptable pressure state based on the plurality of the pressure readings, the threshold requirement includes a step threshold requirement and a percentage threshold requirement, the step threshold requirement is not satisfied when the rolling step count exceeds a step count threshold and the percentage threshold requirement is not satisfied when the percentage of time spent in the unacceptable pressure state exceeds a percentage threshold, and the plurality of information alerts includes a combination information alert.

In some examples, the plurality of sensor readings includes a plurality of accelerometer readings and a plurality of temperature readings, the one or more comparison metrics includes a rolling step count, a first temperature difference, and a second temperature difference, the threshold requirement includes a step threshold requirement and a temperature threshold requirement, the step threshold requirement is not satisfied when the rolling step count exceeds a step count threshold and the temperature threshold requirement is not satisfied when the first temperature difference and the second temperature difference exceed a temperature threshold, and the plurality of information alerts includes a combination information alert.

In some examples, the input device is footwear.

In some examples, the input device is an insole or a pair of insoles.

According to some aspects, there is provided a non-transitory computer readable medium storing computer-executable instructions, which when executed by a computer processor, cause the computer to carry out a method of generating a risk score. The method includes: for each user in a plurality of users, collecting a plurality of sensor readings over a time period using an input device worn by the user, the input device configured to collect the plurality of sensor readings; calculating one or more comparison metrics based on the plurality of sensor readings; determining whether the one or more comparison metrics satisfies one or more threshold requirements; generating a plurality of information alerts if the one or more comparison metrics does not satisfy the one or more threshold requirements in the time period, each information alert corresponding to an information alert type; generating a risk factor for each information alert based on the information alert type; generating one or more alert totals for the time period based on the plurality of information alerts; generating a risk score for the user based on each of the risk factors and the one or more alert totals; and ordering the plurality of users in a list according to the risk score for each user.

A method for generating a risk factor is disclosed. In some aspects, a method for generating a risk factor includes collecting a plurality of pressure readings using an input device; collecting a plurality of temperature readings using the input device; collecting a plurality of accelerometer readings using the input device, the input device worn by a user and configured to collect the plurality of pressure readings, the plurality of temperature readings, and the plurality of accelerometer readings; determining a usage time of the input device for a first time period by identifying and summing time periods when the plurality of pressure readings, the plurality of temperature readings, and the plurality of accelerometer readings have non-baseline values; and generating a risk factor, wherein the risk factor is medium-high when the usage time is below a usage threshold.

In some examples, the method further includes generating an action alert based on the risk factor.

In some examples, the method further includes presenting the action alert.

In some examples, the usage threshold is 4.5 hours.

In some examples, the action alert includes an engagement incentivization alert and/or a foot examination alert when the risk factor is medium-high.

In some examples, the risk factor is low when a percentage of time spent in an unacceptable pressure state based on the plurality of the pressure readings exceeds a percentage threshold in a second time period.

In some examples, the percentage of time spent in the unacceptable pressure state is determined using a pressure threshold, and the pressure threshold is 50 mmHg.

In some examples, the percentage threshold is in a range of 40% to 50%.

In some examples, the action alert includes an activity modification alert, a pressure offloading alert, a custom orthotic alert, a surgical alert, and/or a foot examination alert when the risk factor is low.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state is associated with a second metatarsal of a foot of the user.

In some examples, the orthotic recommendation is a single dancer pad or a double dancer pad when the percentage of time spent in the unacceptable pressure state is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is medium when a first temperature difference based on the plurality of temperature readings and a second temperature difference based on the plurality of temperature readings exceed a temperature threshold.

In some examples, the first temperature difference is determined by comparing a first temperature reading at a first plantar location to a first temperature reading at a second plantar location for a first time, and the second temperature difference is determined by comparing a second temperature reading at the first plantar location to a second temperature reading at the second plantar location for a second time.

In some examples, the temperature threshold is at least 2.0 degrees Celsius.

In some examples, the action alert includes an activity modification alert, a pressure offloading alert, a custom orthotic alert, a vascular assessment requirement alert, and/or a foot examination alert when the risk factor is medium.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the first plantar location or the second plantar location is a second metatarsal of a foot of the user.

In some examples, the orthotic recommendation is a single dancer pad or a double dancer pad when the first plantar location or the second plantar location is a metatarsal head of a foot of the user.

In some examples, the method further includes computing a rolling step count for a third time period based on the plurality of accelerometer readings.

In some examples, the risk factor is high when a percentage of time spent in an unacceptable pressure state based on the plurality of the pressure readings exceeds a percentage threshold in a second time period and the rolling step count for the third time period exceeds a percentage of a step count baseline, or when a first temperature difference based on the plurality of temperature readings and a second temperature difference based on the plurality of temperature readings exceed a temperature threshold and the rolling step count for the third time period exceeds the percentage of the step count baseline.

In some examples, the step count baseline is an average step count of a plurality of previous time periods.

In some examples, the percentage of the step count baseline is 50%.

In some examples, the action alert includes an activity modification alert, a pressure offloading alert, a custom orthotic alert, a surgical alert, a vascular assessment requirement alert, and/or a foot examination alert when the risk factor is high.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state, the first temperature difference, or the second temperature difference is associated with a second metatarsal of a foot of the user.

In some examples, the orthotic recommendation is a single dancer pad or a double dancer pad when the percentage of time spent in the unacceptable pressure state, the first temperature difference, or the second temperature difference is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is zero when: a percentage of time spent in an unacceptable pressure state based on the plurality of the pressure readings does not exceed a percentage threshold in a second time period; a first temperature difference and/or a second temperature difference does not exceed a temperature threshold; the usage time is above the usage threshold; and the rolling step count for the third time period does not exceed the percentage of the step count baseline.

In some examples, the input device is footwear.

In some examples, the input device is an insole or a pair of insoles.

A system for generating a risk factor is disclosed. In some aspects, a system for generating a risk factor includes an input device worn by a user, the input device including: one or more pressure sensors, the one or more pressure sensors configured to collect a plurality of pressure readings; two or more temperature sensors, the two or more temperature sensors configured to collect a plurality of temperature readings; an accelerometer, the accelerometer configured to collect a plurality of accelerometer readings; a processor in communication with the input device, the processor configured to: receive the plurality of pressure readings from the input device; receive the plurality of temperature readings from the input device; receive the plurality of accelerometer readings from the input device; determine a usage time of the input device for a first time period by identifying and summing time periods when the plurality of pressure readings, the plurality of temperature readings, and the plurality of accelerometer readings have non-baseline values; and generate a risk factor, wherein the risk factor is medium-high when the usage time is below a usage threshold.

In some examples, the processor is further configured to generate an action alert based on the risk factor.

In some examples, the system further includes a display, the display configured to present the action alert.

In some examples, the usage threshold is 4.5 hours.

In some examples, the action alert includes an engagement incentivization alert and/or a foot examination alert when the risk factor is medium-high.

In some examples, the risk factor is low when a percentage of time spent in an unacceptable pressure state based on the plurality of the pressure readings exceeds a percentage threshold in a second time period.

In some examples, the percentage of time spent in the unacceptable pressure state is determined using a pressure threshold, and the pressure threshold is 50 mmHg.

In some examples, the percentage threshold is in a range of 40% to 50%.

In some examples, the action alert includes an activity modification alert, a pressure offloading alert, a custom orthotic alert, a surgical alert, and/or a foot examination alert when the risk factor is low.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state is associated with a second metatarsal of a foot of the user.

In some examples, the orthotic recommendation is a single dancer pad or a double dancer pad when the percentage of time spent in the unacceptable pressure state is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is medium when a first temperature difference based on the plurality of temperature readings and a second temperature difference based on the plurality of temperature readings exceed a temperature threshold.

In some examples, the processor is further configured to: determine the first temperature difference by comparing a first temperature reading at a first plantar location to a first temperature reading at a second plantar location for a first time; and determine the second temperature difference by comparing a second temperature reading at the first plantar location to a second temperature reading at the second plantar location for a second time.

In some examples, the temperature threshold is at least 2.0 degrees Celsius.

In some examples, the action alert includes an activity modification alert, a pressure offloading alert, a custom orthotic alert, a vascular assessment requirement alert, and/or a foot examination alert when the risk factor is medium.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the first plantar location or the second plantar location is a second metatarsal of a foot of the user.

In some examples, the orthotic recommendation is a single dancer pad or a double dancer pad when the first plantar location or the second plantar location is a metatarsal head of a foot of the user.

In some examples, the processor is further configured to compute a rolling step count for a third time period based on the plurality of accelerometer readings.

In some examples, the risk factor is high when a percentage of time spent in an unacceptable pressure state based on the plurality of the pressure readings exceeds a percentage threshold in a second time period and the rolling step count for the third time period exceeds a percentage of a step count baseline, or when a first temperature difference based on the plurality of temperature readings and a second temperature difference based on the plurality of temperature readings exceed a temperature threshold and the rolling step count for the third time period exceeds the percentage of the step count baseline.

In some examples, the step count baseline is an average step count of a plurality of previous time periods.

In some examples, the percentage of the step count baseline is 50%.

In some examples, the action alert includes an activity modification alert, a pressure offloading alert, a custom orthotic alert, a surgical alert, a vascular assessment requirement alert, and/or a foot examination alert when the risk factor is high.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state, the first temperature difference, or the second temperature difference is associated with a second metatarsal of a foot of the user.

In some examples, the orthotic recommendation is a single dancer pad or a double dancer pad when the percentage of time spent in the unacceptable pressure state, the first temperature difference, or the second temperature difference is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is zero when: a percentage of time spent in an unacceptable pressure state based on the plurality of the pressure readings does not exceed a percentage threshold in a first time period; a first temperature difference and/or a second temperature difference do not exceed the temperature threshold; the usage time is above the usage threshold; and the rolling step count for the third time period does not exceed the percentage of the step count baseline.

In some examples, the input device is footwear.

In some examples, the input device is an insole or a pair of insoles.

A method for generating a risk factor is disclosed. In some aspects, a method for generating a risk factor includes collecting a plurality of pressure readings using an input device worn by a user; collecting a plurality of temperature readings using the input device; collecting a plurality of accelerometer readings using the input device; computing a rolling step count for a first time period based on the plurality of accelerometer readings; generating a risk factor, wherein the risk factor is high when a percentage of time spent in an unacceptable pressure state based on the plurality of the pressure readings exceeds a percentage threshold in a second time period and the rolling step count for the first time period exceeds a percentage of a step count baseline, or when a first temperature difference based on the plurality of temperature readings and a second temperature difference based on the plurality of temperature readings exceed a temperature threshold and the rolling step count for the first time period exceeds the percentage of the step count baseline.

In some examples, the method further includes generating an action alert based on the risk factor.

In some examples, the method further includes presenting the action alert.

In some examples, the step count baseline is an average step count of a plurality of previous time periods.

In some examples, the percentage of the step count baseline is 50%.

In some examples, the action alert includes an activity modification alert, a pressure offloading alert, a custom orthotic alert, a surgical alert, a vascular assessment requirement alert, and/or a foot examination alert when the risk factor is high.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state, the first temperature difference, or the second temperature difference is associated with a second metatarsal of a foot of the user.

In some examples, the orthotic recommendation is a single dancer pad or a double dancer pad when the percentage of time spent in the unacceptable pressure state, the first temperature difference, or the second temperature difference is associated with a metatarsal head of a foot of the user.

In some examples, the surgical alert includes a surgical relief cut recommendation when the percentage of time spent in the unacceptable pressure state is associated with a midfoot of a foot of the user.

In some examples, the method further includes determining a usage time of the input device for a third time period by identifying and summing time periods when the plurality of pressure readings, the plurality of temperature readings, and the plurality of accelerometer readings have non-baseline values.

In some examples, the risk factor is medium-high when the usage time is below a usage threshold.

In some examples, the usage threshold is 4.5 hours.

In some examples, the action alert includes an engagement incentivization alert and/or a foot examination alert when the risk factor is medium-high.

In some examples, the risk factor is low when the percentage of time spent in the unacceptable pressure state exceeds the percentage threshold in the second time period.

In some examples, the percentage of time spent in the unacceptable pressure state is determined using a pressure threshold, and the pressure threshold is 50 mmHg. In some examples, the percentage threshold is in a range of 40% to 50%.

In some examples, the action alert includes an activity modification alert, a pressure offloading alert, a custom orthotic alert, a surgical alert, and/or a foot examination alert when the risk factor is low.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state is associated with a second metatarsal of a foot of the user.

In some examples, the orthotic recommendation is a single dancer pad or a double dancer pad when the percentage of time spent in the unacceptable pressure state is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is medium when the first temperature difference based on the plurality of temperature readings and the second temperature difference based on the plurality of temperature readings exceed the temperature threshold.

In some examples, the first temperature difference is determined by comparing a first temperature reading at a first plantar location to a first temperature reading at a second plantar location for a first time, and the second temperature difference is determined by comparing a second temperature reading at the first plantar location to a second temperature reading at the second plantar location for a second time.

In some examples, the temperature threshold is at least 2.0 degrees Celsius.

In some examples, the action alert includes an activity modification alert, a pressure offloading alert, a custom orthotic alert, a vascular assessment requirement alert, and/or a foot examination alert when the risk factor is medium.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the first plantar location or the second plantar location is a second metatarsal of a foot of the user.

In some examples, the orthotic recommendation is a single dancer pad or a double dancer pad when the first plantar location or the second plantar location is a metatarsal head of a foot of the user.

In some examples, the risk factor is zero when: the percentage of time spent in the unacceptable pressure state does not exceed the percentage threshold in the second time period; the first temperature difference and/or the second temperature difference does not exceed the temperature threshold; the usage time is above the usage threshold; and the rolling step count for the first time period does not exceed the percentage of the step count baseline.

In some examples, the input device is footwear.

In some examples, the input device is an insole or a pair of insoles.

A system for generating a risk factor is disclosed. In some aspects, a system for generating a risk factor includes an input device worn by a user, the input device including; one or more pressure sensors, the one or more pressure sensors configured to collect a plurality of pressure readings; two or more temperature sensors, the two or more temperature sensors configured to collect a plurality of temperature readings; an accelerometer, the accelerometer configured to collect a plurality of accelerometer readings; a processor in communication with the input device, the processor configured to: receive the plurality of pressure readings from the input device; receive the plurality of temperature readings from the input device; receive the plurality of accelerometer readings from the input device; compute a rolling step count for a first time period based on the plurality of accelerometer readings.

In some examples, the processor is further configured to generate an action alert based on the risk factor.

In some examples, the system further includes a display, the display configured to present the action alert.

In some examples, the step count baseline is an average step count of a plurality of previous time periods.

In some examples, the percentage of the step count baseline is 50%.

In some examples, the action alert includes an activity modification alert, a pressure offloading alert, a custom orthotic alert, a surgical alert, a vascular assessment requirement alert, and/or a foot examination alert when the risk factor is high.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state, the first temperature difference, or the second temperature difference is associated with a second metatarsal of a foot of the user.

In some examples, the orthotic recommendation is a single dancer pad or a double dancer pad when the percentage of time spent in the unacceptable pressure state, the first temperature difference, or the second temperature difference is associated with a metatarsal head of a foot of the user.

In some examples, the surgical alert includes a surgical relief cut recommendation when the percentage of time spent in the unacceptable pressure state is associated with a midfoot of a foot of the user.

In some examples, the processor is further configured to determine a usage time of the input device for a third time period by identifying and summing time periods when the plurality of pressure readings, the plurality of temperature readings, and the plurality of accelerometer readings have non-baseline values.

In some examples, the risk factor is medium-high when the usage time is below a usage threshold.

In some examples, the usage threshold is 4.5 hours.

In some examples, the action alert includes an engagement incentivization alert and/or a foot examination alert when the risk factor is medium-high.

In some examples, the risk factor is low when the percentage of time spent in the unacceptable pressure state exceeds the percentage threshold in a second time period.

In some examples, the percentage of time spent in the unacceptable pressure state is determined using a pressure threshold, and the pressure threshold is 50 mmHg.

In some examples, the percentage threshold is in a range of 40% to 50%.

In some examples, the action alert includes an activity modification alert, a pressure offloading alert, a custom orthotic alert, a surgical alert, and/or a foot examination alert when the risk factor is low.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the percentage of time spent in the unacceptable pressure state is associated with a second metatarsal of a foot of the user.

In some examples, the orthotic recommendation is a single dancer pad or a double dancer pad when the percentage of time spent in the unacceptable pressure state is associated with a metatarsal head of a foot of the user.

In some examples, the surgical alert includes a surgical relief cut recommendation when the percentage of time spent in the unacceptable pressure state is associated with a midfoot of a foot of the user.

In some examples, the risk factor is medium when the first temperature difference based on the plurality of temperature readings and the second temperature difference based on the plurality of temperature readings exceed the temperature threshold.

In some examples, is further configured to: determine the first temperature difference by comparing a first temperature reading at a first plantar location to a first temperature reading at a second plantar location for a first time; and determine the second temperature difference by comparing a second temperature reading at the first plantar location to a second temperature reading at the second plantar location for a second time.

In some examples, the temperature threshold is at least 2.0. degrees Celsius.

In some examples, the action alert includes an activity modification alert, a pressure offloading alert, a custom orthotic alert, a vascular assessment requirement alert, and/or a foot examination alert when the risk factor is medium.

In some examples, the custom orthotic alert includes an orthotic recommendation. The orthotic recommendation may be a single dancer pad, a double dancer pad, a metatarsal bar, a metatarsal pad, a u-shaped metatarsal pad, an extrinsic post, or a heel lift.

In some examples, the orthotic recommendation is a metatarsal pad or a u-shaped metatarsal pad when the first plantar location or the second plantar location is a second metatarsal of a foot of the user.

In some examples, the orthotic recommendation is a single dancer pad or a double dancer pad when the first plantar location or the second plantar location is a metatarsal head of a foot of the user.

In some examples, the risk factor is zero when: the percentage of time spent in the unacceptable pressure state does not exceed the percentage threshold in the first time period; the first temperature difference and/or a second temperature difference do not exceed the temperature threshold; the usage time is above the usage threshold; and the rolling step count for the first time period does not exceed the percentage of the step count baseline.

In some examples, the input device is footwear.

In some examples, the input device is an insole or a pair of insoles.

According to some aspects, there is provided a non-transitory computer readable medium storing computer-executable instructions, which when executed by a computer processor, cause the computer to carry out a method of generating a risk factor. The method includes: collecting a plurality of pressure readings using an input device worn by a user; collecting a plurality of temperature readings using the input device; collecting a plurality of accelerometer readings using the input device; computing a rolling step count for a first time period based on the plurality of accelerometer readings; and generating a risk factor, wherein the risk factor is high when a percentage of time spent in an unacceptable pressure state based on the plurality of pressure readings exceeds a percentage threshold in a second time period and the rolling step count for the first time period exceeds a percentage of a step count baseline, or when a first temperature difference based on the plurality of temperature readings and a second temperature difference based on the plurality of temperature readings exceed a temperature threshold and the rolling step count for the first time period exceeds the percentage of the step count baseline.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example input device usable in the methods and systems described herein;

FIG. 2 is an exploded view of the input device of FIG. 1;

FIG. 3 is a flow chart illustrating a method of generating a risk score for a plurality of users, which may be carried out using the input device of FIG. 1;

FIG. 4 is a flow chart illustrating a method of generating a risk factor that may be used with the method of FIG. 3;

FIG. 5 is a flow chart illustrating an example of the method of FIG. 4, using pressure readings collected using the input device of FIG. 1;

FIG. 6 is a flow chart illustrating an example of the method of FIG. 4, using temperature readings collected using the input device of FIG. 1;

FIG. 7 is a flow chart illustrating an example of the method of FIG. 4, using sensor readings collected from various sensors using the input device of FIG. 1;

FIG. 8 is a flow chart illustrating an example of the method of FIG. 4, using accelerometer readings collected using the input device of FIG. 1;

FIG. 9 is a flow chart illustrating an example of the method of FIG. 4, using pressure, temperature, and accelerometer readings collected using the input device of FIG. 1; and

FIG. 10 is a diagram illustrating a risk factor hierarchy for information alerts generated from pressure, temperature, and accelerometer readings collected using the input device of FIG. 1.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus, process, or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements.

Numerous specific details are set forth herein in order to provide a thorough understanding of the subject matter described herein. However, the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of A and B” is intended to mean only A (i.e. one or multiple of A), only B (i.e. one or multiple of B), or a combination of one or more of A and one or more of B.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

As will be described in further detail below, described herein are methods and systems for generating a risk score for each user in a plurality of users, using an input device worn on the foot of the user. The risk score indicates the user's risk of developing a diabetic foot ulcer. As used herein, the term “worn” (and related terms such as “wearable” and “wear”) indicates that the referenced part may be affixed to, adhered to, placed on, or placed in the user's body, or affixed to, adhered to, placed on, placed in, placed in proximity to, or integral with the user's clothing. Such clothing may include but is not limited to a shoe, a sock, an insole, and/or another type of foot-worn clothing (including custom orthotics or generic insoles).

The sensors described herein can include pressure sensors. As used herein, the term “pressure” is used broadly and can refer to raw force (i.e. with units of N), or pressure resulting from a raw force (i.e. with units of N/m2). The pressure readings acquired by the pressure sensors can be used to determine the level of pressure applied by an individual's foot when performing various activities such as walking, running, sliding or jumping for example.

The systems, and methods described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Generally disclosed herein are systems and methods for generating a risk score for each user in a plurality of users. The risk score indicates the user's risk of developing a diabetic foot ulcer. Systems for generating a risk score may include an input device with sensors, and the sensors on the input device may include a plurality of pressure sensors, a plurality of temperature sensors, and an accelerometer, which are used to obtain a plurality of pressure readings, temperature readings, and accelerometer readings, respectively. In particular, the plurality of pressure sensors, the plurality of temperature sensors, and the accelerometer may be worn on a user's foot. In some particular examples, the sensors are included in an input device in the form of an insole (or alternatively, a pair of insoles) which may be inserted into a user's shoe and worn underfoot. The input device may in turn be part of a system, which may further include a non-transitory storage memory storing a risk score generation method using any of the plurality of pressure readings, the plurality of temperature readings, and the plurality of accelerometer readings to generate information alerts, and to generate a risk score from the information alerts.

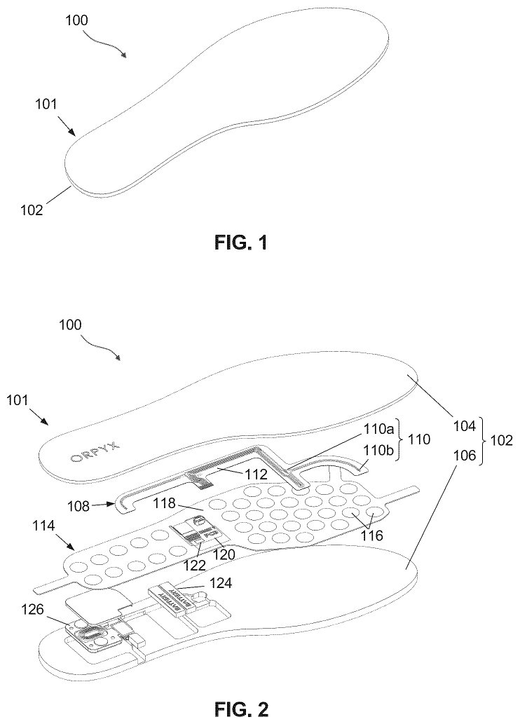

Referring to FIGS. 1 and 2, an example input device 100 in the form of an insole 101 is shown. The insole 101 may be worn in direct contact with the user's skin, or may be spaced from the user's skin (e.g. by a sock). Insoles usable in the processes and systems described herein may be of a variety of configurations, of which the insole 101 is but one example. For example, the insoles may be those available from Orpyx Medical Technologies Inc. (Calgary, Canada) under the brand name Orpyx SI®. For further example, the insoles may be those described in U.S. Pat. No. 10,004,428 (Everett et al.), International Patent Application Publication No. WO/2021/092676 A1 (Everett et al.), U.S. Pat. No. 11,700,904 (Stevens et al.), and/or United States Patent Application Publication No. 2023/0189918 (Stevens et al.). Each of the aforementioned documents is incorporated herein by reference in its entirety.

Referring first to FIG. 1, in the example shown, the input device 100, which can be insole 101, includes an insole bulk 102, which may be made up of one or more layers such as a cushion layer, a support layer, a gel layer, an anti-odor layer, a thermal insulation layer, and/or a foam layer. In the example shown, the input device 100 is in the form of a generic insole that can be used by a variety of users and in a variety of footwear. The insole bulk 102 includes a top layer 104 and a base layer 106 (shown in FIG. 2). The top layer 104 may in turn include multiple sub-layers, such as an upper finishing layer (not shown), a middle comfort layer (not shown), and a contoured layer (not shown). Likewise, the base layer may include multiple sub-layers (not shown).

In other examples, the insole may be a different type of generic insole (e.g. a comfort insole, a flat insole, an athletic insole, a shock-absorbing insole, or a gel insole), or the insole may be an orthotic that is custom manufactured for a user. For an orthotic that is custom manufactured for a user, the user's foot may be assessed (e.g. by a podiatrist, optionally using plaster casting or 3D scanning), and the insole bulk 102 may be custom fashioned based on the assessment, for example in order to support the user's foot, improve foot function, relieve pain, and/or relieve pressure. Furthermore, the insole may in some examples be integral with a shoe. The insole may be available in different sizes.

Referring to FIG. 2, in the example shown, a temperature sensor array 108 is embedded in the insole bulk 102. The temperature sensor array 108 includes two or more temperature sensors 110 (i.e. a first temperature sensor 110a, a second temperature sensor 110b, and so on, only two of which are labelled) printed on flexible polymer film 112. The temperature sensor 110 or the temperature sensor array 108 may be printed directly onto the insole 101 or a portion of the insole 101. The temperature sensors 110 may be, for example, multilayer chip thermistors, and may have an accuracy of at least 0.2 degrees Celsius. The temperature sensors 110 may be configured to serially obtain measurements, for example continuously at a frequency, or intermittently at regular intervals, or sporadically.

As used herein, the term ‘temperature measurements’ refers to the raw data generated by the temperature sensors 110. These temperature measurements may be processed downstream, for example by a processor as described below, to yield ‘processed temperature measurements’. The term ‘temperature readings’ is used herein to generally refer to the ‘temperature measurements’, and/or the ‘processed temperature measurements’. That is, the term ‘temperature readings’ may refer to temperature measurements, processed temperature measurements, or a combination thereof. The term ‘series of temperature readings’ refers to temperature readings relating to measurements taken in series over a time window.

Referring still to FIG. 2, the input device 100 includes a pressure sensor array 114 embedded in the insole bulk 102. The pressure sensor array 114 includes one or more pressure sensors 116 (only two of which are labelled) printed on flexible polymer film 118.

Each pressure sensors 116 may obtain continuous or frequent interval measurements and may produce a series of measurements. A higher pressure sensor data rate (e.g. 64 Hz, 128 Hz or 256 Hz) may in some examples be preferred, to reduce pre-process noise occurring during data down sampling. The pressure sensors 116 may be flat and flexible. For example, the pressure sensors 116 may be force sensing resistors, pressure sensors, piezoelectric tactile sensors, elasto-resistive sensors, capacitive sensors or more generally, any type of force sensor that can be integrated into an input device such as insole 101. The number of pressure sensors 116 may depend on the size of the user's foot and may be determined by a set metric relating to the subject's foot. For example, the pressure sensors 116 may cover 25% of the surface area of the subject's foot. The pressure sensors 116 may also cover critical areas of the subject's foot. For example, the force sensors 110 may be denser in the metatarsal and heel areas.

As used herein, the term ‘pressure measurements’ is used herein to refer to the raw data generated by the pressure sensors 116. These pressure measurements may be processed downstream (e.g. normalized and/or standardized, such as normalized by the bodyweight of the subject), for example by a processor as described below, to yield ‘processed pressure measurements’. The term ‘pressure readings’ is used herein to generally refer to the ‘pressure measurements’, and/or the ‘processed pressure measurements’. That is, the term ‘pressure readings’ may refer to pressure measurements, processed pressure measurements, or a combination thereof. The term ‘series of pressure readings’ refers to pressure readings relating to measurements taken in series over a time window.

In the example shown, the input device 100 further includes a first inertial measurement unit (IMU) 122. In the example shown, the IMU 122 is integral with processor 120 (described below), and is shown schematically. The IMU 122 can include one or more sensors for measuring the position and/or motion of the subject. For example, the IMU 122 may include one or more of a gyroscope, accelerometer (e.g., a three-axis accelerometer), magnetometer, orientation sensor (for measuring orientation and/or changes in orientation), angular velocity sensor, and inclination sensor. Generally, the IMU 122 includes at least an accelerometer. The IMU 122 may be included in any section of the insole bulk 102 or may be external to the insole bulk 102. For example, the IMU 114 may be included in the user's shoe or clipped to the user's shoelaces. More than one IMU may be included within or external to the insole bulk 102 to provide more comprehensive information.

As used herein, the term ‘accelerometer measurements’ is used herein to refer to the raw accelerometer data generated by the IMU 122. These accelerometer measurements may be processed downstream, for example by a processor 120 as described below, to yield ‘processed accelerometer measurements’. The term ‘accelerometer readings’ is used herein to generally refer to the ‘accelerometer measurements’, and/or the ‘processed accelerometer measurements’. That is, the term ‘accelerometer readings’ may refer to accelerometer measurements, processed accelerometer measurements, or a combination thereof. The term ‘series of accelerometer readings’ refers to accelerometer readings relating to measurements taken in series over a time window.

Referring still to FIG. 2, the input device 100 further includes a processor 120 that receives force measurements from the pressure sensors 116, temperature measurements from the temperature sensors 110, accelerometer measurements from the IMU 122 (and optionally additional measurements from optional supplemental sensors). Measurements from the pressure sensors 116, temperature sensors 110, and IMU 122 may include raw data such as resistance data, capacitance data, and/or other raw data. The processor 120 may apply an algorithm for reducing data noise, and/or for scaling/calibrating the measurements, to yield processed pressure measurements, processed temperature measurements, and processed accelerometer measurements. Communication between the processor 120 and the various sensors may be through any wired or wireless connection.

In an alternative example, the pressure measurements, temperature measurements and/or accelerometer measurements may be transmitted to a remote processor (not shown) for processing (i.e. it is possible for no processing or for minimal processing to occur within the input device 100). The remote processor may be located on any suitable mobile or other device (e.g. smartphone, smartwatch, tablet, laptop computer, desktop computer, cloud-based server etc.). The remote processor may be used in addition to the processor 120 and may provide additional processing resources not available via the processor 120, or may be used instead of the processor 120.

One example of a remote processor is a cloud server, which may be used to provide additional processing capabilities. For example, some aspects of processing may be delegated to the cloud server to conserve power resources in the input device 100. The cloud server may store the pressure readings, temperature readings, and accelerometer readings and/or allow for more complex processing of the pressure readings, temperature readings, and accelerometer readings. In some cases, the cloud server, processor 120 and/or other remote processor may communicate in real-time to provide timely feedback to the subject.

Additionally, the pressure readings, temperature readings, and/or accelerometer readings may be transmitted through an optional external relay device (e.g. cellphone or wall hub), before being transmitted to the cloud server through a wired or wireless connection. The external relay device may store the pressure, temperature, and/or the accelerometer readings until transmission to a remote cloud server or output device is available. Additionally, the external relay device may be included in the remote processor device.

As will be described below, the processor 120 and/or remote processor may input pressure, temperature, and accelerometer readings into a remote patient monitoring program, which is stored in the memory of the system. The storage and/or memory of the system may also store the sensor data, including pressure readings, temperature readings, accelerometer readings, alerts, risk factors, risk scores, and any other data, algorithm, and/or equation that may be used to generate the risk score, as discussed below.

Referring still to FIG. 2, a pair of batteries 124 are further embedded in the insole bulk 102, for providing energy to the components of the input device 100. Alternatively, a single battery (not shown) may be embedded in the insole 101 for providing energy to the components of the insole 101. In the shown example, the batteries 124 can be charged by a wireless charging assembly, which includes a wireless charging transmitter (not shown) and a wireless charging receiver 126 that is embedded in the insole bulk 102. Alternatively, the batteries may be charged by a wireless charging assembly including one wireless transmitter external to the input device 100 and more than one wireless charging receiver 126, wherein each input device 100 includes one wireless charging receiver. Alternatively, the battery or batteries may be non-rechargeable (not shown), wherein the wireless charging receiver 126 is omitted.

As noted above, various supplemental sensors may be included in the input device 100, for generating supplemental measurements. Such sensors may include one or more of a GPS sensor, a heart rate sensor, a respiratory rate sensor, a blood pressure sensor, a blood oxygen saturation sensor, a blood flow sensor, a blood or environmental content quantification sensor (e.g. glucose, electrolytes, minerals, oxygen, carbon dioxide, carbon monoxide, HbAlC, Ethanol, protein, lipid, carbohydrate, cortisol, lactate, pH, pro-and anti-inflammatory markers, MMPs, growth factors, bacterial content), a hydration status/tissue turgor sensor, a joint position sensor, a gait analysis sensor (including supination and pronation), a device breakdown sensor, a pedometer, an accelerometer, a velocity sensor, a calorimetry sensor, a centre of gravity sensor, a centre of foot position sensor, a friction sensor, a traction sensor, a contact area sensor, a connectivity/insulation sensor, an EEG sensor, a barometer, an altimeter, and/or an ECG sensor.

Optionally, the input device 100 may include one or more stimulators (not shown) for providing feedback to the user. The stimulator(s) may provide a tactile (e.g. vibratory), audible, or visual stimulus to the user.

The components of the input device 100 may be generally flat, and/or may be nested within pockets of the insole bulk 102, so that in use the user generally does not feel the presence of the input device 100 components under their foot.

In the example shown, the input device is in the form of a single insole 101; however, in alternative examples, the input device may be in the form of a pair of insoles (i.e. a left insole and a right insole, which can be worn concurrently).

In further alternative examples, the pressure sensors, temperature sensors, and IMU may be worn on the subject's foot in another fashion other than via an insole.

In further alternative examples, the pressure sensors, temperature sensors, and IMU may not be worn on the subject's foot such as via the insole 101, but may be disposed in a non-wearable input device with which the subject's foot comes into contact, such as a mat, a platform, a treadmill, or equipment.

As mentioned above, the input device 100 may be part of a system that includes an output device (not shown). The output device may be, for example, a display screen, a speaker, or a tactile stimulator (e.g. a vibratory device). The input device 100 may be in communication with the output device (optionally in direct communication, or in communication via a relay device, cloud server, or remote processing device). The output device may be configured to output an indication of pressure readings, temperature readings, accelerometer readings, alerts, risk factors, alert totals, or risk scores, as described in further detail below. The output device may be configured to display to the user of the input device or to additional parties.

Methods for generating a risk score for a user will now be described. For clarity, the methods will be described with reference to input device 100 in the form of insole 101; however, the methods are not limited to an insole. In general, methods for generating a risk score may include: for each user in a plurality of users, collecting a series of sensor readings using a plurality of sensors (e.g. pressure sensors 116) using an input device 100 worn by the user, during a time period; calculating one or more comparison metrics based on the plurality of sensor readings; determining whether the one or more comparison metrics does not satisfies one or more threshold requirements in the time period; generating a plurality of information alerts if the one or more comparison metrics does not satisfy the one or more threshold requirements in the time period; generating a risk factor for information alert based on the information alert type; generating one or more alert totals for the time period based on the plurality of information alerts; generating a risk score for each user in the plurality of users based on each of the risk factors and the one or more alert totals; and ordering the plurality of users in a list according to the risk score for each user. The risk score method may be stored in a non-transitory storage memory, which may be included in the input device 100 or may be remote from the input device 100. A processor (e.g. processor 120 or a remote processor as described above) receives the sensor readings and carries out the risk score generation.

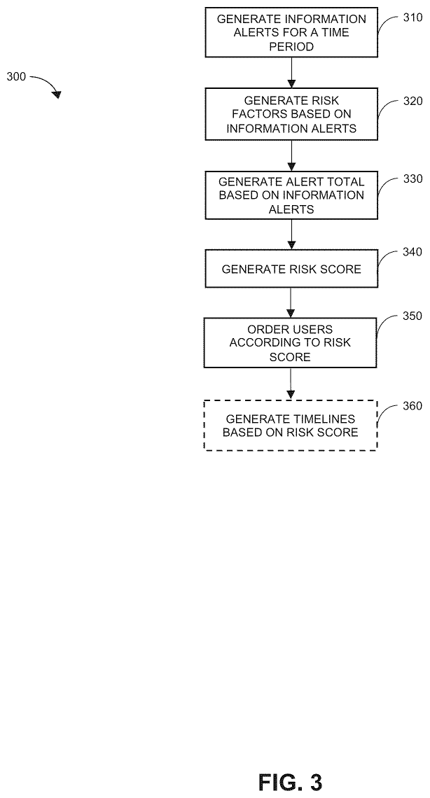

Referring now to FIG. 3, shown therein is a method of generating a risk score. A risk score is a numeric value representing a user's risk of developing a diabetic foot ulcer. Method 300 may be used to generate a risk score for a single user, in order to provide a more detailed understanding of the user's particular risk. Alternatively, method 300 may be used to generate risk scores for multiple users, in order to triage users in order of treatment urgency or risk severity. Method 300 may be repeated to regularly update risk scores as users' circumstances change. Method 300 may be carried out by input device 100.

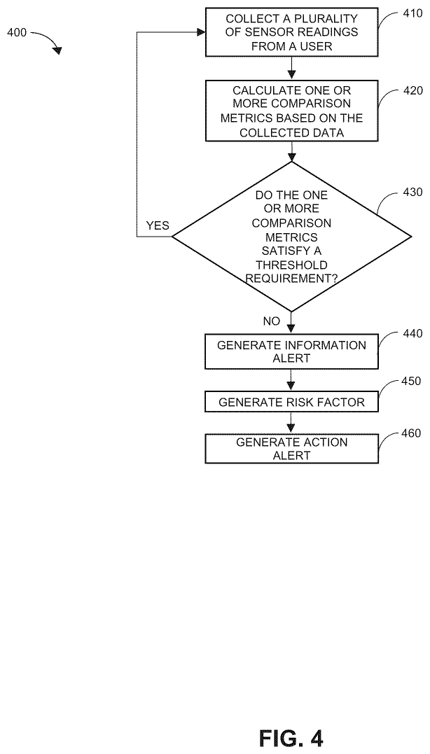

At step 310, information alerts are generated by the processor within a time period. Information alerts are alerts that indicate that a threshold requirement has not been satisfied for a particular type of sensor reading collected by the input device, and that the input device 100 is generating sensor readings in a range that is considered unsafe for the user. Different types of information alerts may be generated based on the types of sensor readings collected by the input device. For example, pressure information alerts (when the input device collects pressure readings), temperature information alerts (when the input device collects temperature readings), adherence alerts (when the input device collects any type of sensor reading), etc. may be generated. Information alerts may be presented or communicated to a user wearing the input device, such that the user can adjust their behavior to clear the alerts and reduce their tissue ulceration risk. Information alerts may be generated on a regular frequency, such as once per day. Information alerts may be regenerated if they are not addressed in a timely manner. The time period for which the information alerts are generated at step 310 may be longer than the information alert generation frequency. For example, if information alerts are generated daily, the time period may be one month. Therefore, multiple information alerts, and multiple types of information alerts may be captured within the time period. A method for generating a risk factor is shown in FIG. 4 and is described in further detail below, and the method includes steps for generating information alerts.

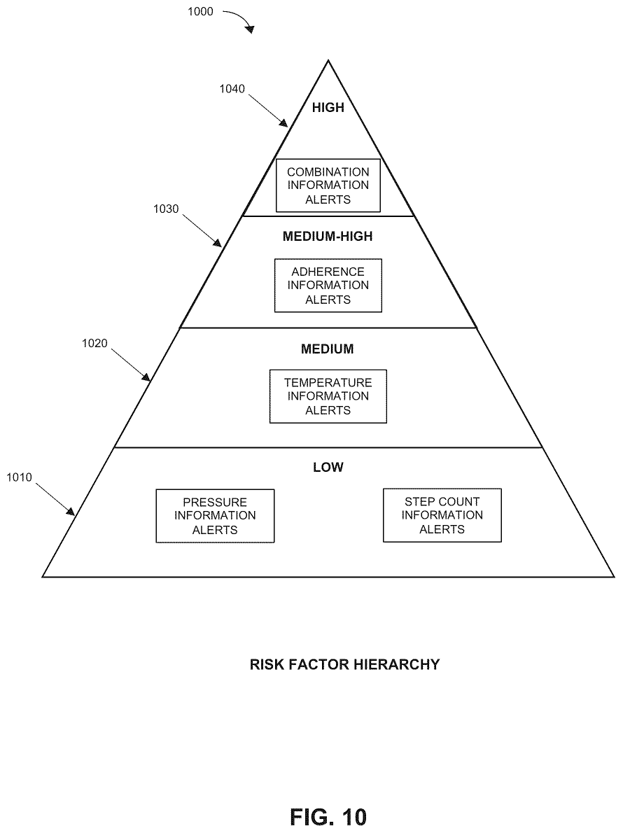

At step 320, risk factors are generated based on the information alerts generated at step 310. Risk factors are levels of concern that are associated with the types of information alerts generated. Different information alert types may indicate different ulceration stages for a user, and therefore there are varying levels of concern for the different information alert types. For example, the processor 120 or the remote processor may generate a pressure information alert, and pressure information alert may indicate that inflammation may occur in the near future for a user. The processor 120 or the remote processor may also generate a temperature information alert, and temperature information alert may indicate that inflammation and tissue damage are actively occurring for the user. Therefore, a temperature information alert would be considered indicative of a higher ulceration risk for the user, and a higher risk factor would be generated for the temperature information alert than the pressure information alert. An example of a risk factor hierarchy is shown in FIG. 10 and is described in further detail below. Risk factors may be generated as risk factor qualitative levels, such as “low”, “medium”, “medium-high”, or “high”, or they may be generated as risk factor quantitative levels, such as numbers on a scale from 1 to 4. Risk factor qualitative levels may be associated with risk factor quantitative levels. For example, “low” risk may be associated with a risk factor quantitative level of 1, “medium” risk may be associated with a risk factor quantitative level of 2, “medium-high” risk may be associated with a risk factor quantitative level of 3, and “high” risk may be associated with a risk factor quantitative level of 4. At step 320, a risk factor is generated for each information alert generated within the time period at step 310. A method for generating a risk factor is shown in FIG. 4 and is described in further detail below.

At step 330, an alert total is generated based on the number of information alerts generated at step 310. An alert total is the summed number of information alerts of all information alert types generated in the time period and/or the summed number of information alerts of a certain information alert type generated in the time period. If multiple information alerts are generated within the time period, this may indicate that the user continues to perform activities that put them in “unsafe” data zones, or that efforts the user takes to mitigate the information alerts and reduce their risk may be ineffective. A greater number of information alerts within the time period likely indicates a higher risk, therefore alert total is an important consideration in the generation of risk score. For example, if 7 alerts are generated for a first user in the time period, the first user should be considered higher risk than a second user for which 2 alerts are generated in the time period. Additionally, an alert total qualitative level may be generated based on the alert total. The alert total qualitative levels may include “low”, “medium”, “medium-high”, and “high” levels. The alert total qualitative levels may be associated with ranges of alert totals (e.g. if the alert total is between 1-3, the alert total qualitative level is “low”, if the alert total is between 4-6, the alert total qualitative level is “medium”, etc.). Additionally or alternatively, an alert total quantitative level may be generated according to the alert total and may include numbers on a scale from 1 to 4. The alert total qualitative levels may be associated with the alert total quantitative levels. The risk factor qualitative levels and the alert total qualitative levels may be the same or different. The risk factor quantitative levels and the alert total quantitative levels may be the same or different.

At step 340, a risk score is generated based on the risk factor and the alert total. The risk score may be calculated using any equation or model that combines the risk factor and the alert total. In one example, the risk score may be calculated as a weighted sum, where each risk factor is associated with a weighting (or the risk factor quantitative level, e.g. WRFLOW=1, WRFMED=2, etc.) that is multiplied by the alert total for each information alert type, as shown in Equation 1:

$$RS = {W}_{{RF}_{1}} * {AT}_{1} + {W}_{{RF}_{2}} * {AT}_{2} + {W}_{{RF}_{3}} * {AT}_{3} + {W}_{{RF}_{4}} * {AT}_{4} \qquad ( 1 )$$

Where RS is the risk score, WRF, is the weighting associated with a risk factor for a first information alert type, AT1 is the alert total for the first information alert type, WRF2 IS the weighting associated with a risk factor for a second information alert type, AT2 is the alert total for the second information alert type, WRF is the weighting associated with a risk factor for a third information alert type, AT3 is the alert total for the third information alert type, WRF, is the weighting associated with a risk factor for a fourth information alert type, and AT4 is the alert total for the fourth information alert type.

Providing an example with Equation 1, if a first user receives 7 pressure information alerts in the time period, and pressure information alerts have a “low” risk factor, and the “low” risk factor provides a weighting of 1, the risk factor that would be generated for the first user according to Equation 1 is 7. For a second patient that receives 2 combination alerts in the time period, and combination alerts have a “high” risk factor, and the “high” risk factor provides a weighting of 4, the risk factor that would be generated for the second user according to Equation 1 is 8. Therefore, the second user, who received fewer alerts in the time period, but whose alerts were associated with a higher risk factor, would have a higher risk score than the first user.

Equation 1 is merely an example of an equation for calculating the risk score from the risk factor and the alert total, but any equation that combines the risk factor and the alert total can be used to generate a risk score. For example, other possible equations and models could include weights, biases, averages, multipliers, etc. Other equations may include the use of risk factor qualitative levels, risk factor quantitative levels, alert total qualitative levels, and/or alert total quantitative levels.

Alternatively, the risk score may be generated using a machine learning model. The machine learning model may be trained to predict a risk score using risk factor training data, alert total training data, and risk score training data, wherein the risk score training data includes risk scores assigned by clinicians.

The risk score may be a numerical value, e.g. provided on a scale from 1 to 10. Alternatively, the risk score may be a qualitative level, such as “low”, “medium”, “medium-high”, and “high”.

The risk factor and the alert total are the main contributors to the risk score calculation at step 340, however additional contributors may be used to generate the risk score.

A first additional contributor that may contribute to the risk score is alert patterns. The processor may search for patterns in the types and numbers of information alerts generated in the time period. For example, the processor may identify a pattern that consists of a pressure information alert (with a “low” risk factor), followed by a temperature information alert (with a “medium” risk factor) within a short period, followed by no alerts for a long period. This particular pattern may indicate that the steps the user is taking in response to the information alerts to offload the pressure on their feet and reduce inflammation are effective. Alternatively, the processor may identify a pattern consisting of a single alert type over a certain number of days (e.g. a temperature information alert over 10 days), which may indicate that the steps the user is taking in response to the information alerts to offload the pressure on their feet and reduce inflammation are ineffective. In a different example, the processor may identify patterns indicative of ulcer progression, such as a pressure information alert (with a “low” risk factor), followed by a temperature alert (with a “medium” risk factor) within a short period, followed by a combination alert (with a “high” risk factor) within another short period. Accordingly, the risk score may be modified by the presence of these patterns. For example, the processor may generate multiplier values if certain patterns are identified, which can be applied to the risk score.

A second additional contributor that may contribute to risk score is foot examination data. Foot examination data may be received by the processor. The foot examination data may indicate a presence or an absence of an abnormality on a foot of a user. The abnormality may be a visual abnormality. The foot examination data may also include a presence or absence of abnormal colors, calluses, foot deformities, and temperature differences that can be determined by touch. Foot examination data may be prompted by a healthcare professional or by the user themselves. In some examples, one or more alerts may prompt observation of the user's foot through a pop-up on the display. The resulting foot examination data may then be used as a second additional contributor to the risk score calculation. The processor may generate numeric values based on the foot examination data to modify the risk score. For example, a presence of an abnormality on a foot of the user may be given a multiplier value of 2, which will double the risk score. The absence of an abnormality on the foot of the user may be associated with a multiplier of 1, which will not affect the risk score. Alternatively, a bias value may be applied to the risk score generation equation or model based on the presence (e.g. +5) or the absence (e.g. +0) of the abnormality. If a presence of an abnormality on a foot of the user is identified, the foot examination data may also include the location data and/or measurement data for the abnormality. The processor may also generate numeric values based on location and/or measurement information. For example, the size of the abnormality may be measured in millimeters and a bias value may be applied to the risk score generation equation or model based on the size (e.g. +5 for a 5 mm abnormality). Alternatively, the processor may assign the foot examination data a qualitative level such as “low”, “medium”, “medium-high”, or “high” risk. The results of a foot examination may be entered on a display and/or automatically inputted from a medical device and transmitted to the processor.

A third additional contributor that may contribute to risk score is scan data. Scan data may include a plurality of sensor readings collected with a scanning device. The scan data may corroborate or refute the information alerts. The risk score may be increased if the information alert is corroborated by the scan data, or it may be decreased if the information alert is refuted by the scan data. For example, the scan data may include a plurality of temperature readings from a thermogram or results from transcutaneous oxygen saturation imaging. If a temperature information alert is generated by the processor based on the temperature readings from the input device, and the processor also receives results of a thermogram that indicate high temperatures in certain plantar locations), the temperature information alert is corroborated by the other examination data, and therefore the risk score should be increased.

A fourth additional contributor that may contribute to risk score is historical user data. The historical user data may capture a user's history of foot issues and other comorbidities, which may impact the user's likelihood of developing future ulcers. Historical user data may include historical ulceration data, historical foot data, historical amputation data, historical foot surgery data, historical social data, nutritional status, historical gait data, historical mobility data, historical medication use, historical comorbidity data, and historical lab test data. Historical ulceration data may include data from previous foot ulcers (e.g. location, measurement, description, infections, etc.). Historical foot data may include foot deformities, foot infections, loss of protective sensation, Charcot foot, bunions, hammer toe, etc. Historical social data may include substance and tobacco use. Historical gait data may include the presence of gait abnormalities (e.g. calcaneal gait), compensatory gait patterns (e.g. early knee flexion, foot or hip abduction, or foot drop), biomechanical issues that cause biomechanical compensation at the foot or ankle (e.g. hip tightness, knee contractures, unilateral weakness, etc.), and anatomical measurements relating to gait (e.g. limb lengths). Historical mobility data may include assistive device (e.g. walkers, canes, etc.) and prescription orthotic use. Historical comorbidity data may include a history of peripheral arterial disease, macrovascular disease, chronic kidney disease, a stroke, an asymmetric neurological disease (e.g. impaired sympathetic tone), muscle mass asymmetry, and edema. Historical user data may also include level of diabetic control. Historical lab test data may be collected by medical equipment, where the historical lab test data specifically indicates a diabetes complication (not including diabetic foot ulcers) that may increase the risk of forming a diabetic foot ulcer. The historical lab test data may include blood test data (e.g. blood glucose or A1C levels), urine test data (e.g. urine albumin-to-creatinine ratio), Semmes-Weinstein monofilament test data (e.g. indicating levels of loss of protective sensation), vascular assessment data, etc. The historical user data may be received by the processor, and the processor may generate numeric values for modifying the risk score. For example, if the patient has had at least one previous diabetic foot ulcers in the past 5 years, a multiplier of 1.5 may be applied to the risk score. If the patient has had at least 5 previous diabetic foot ulcers in the past 5 years, a multiplier of 2 may be applied to the risk score. Numerical values may be assigned to the historical user data by the processor according to clinical standards reflecting known impacts to risk of ulceration. Alternatively, the processor may assign qualitative levels to the historical user data, such as “low”, “medium”, “medium-high”, or “high” risk.

Once the risk score has been generated, actions can be executed based on the risk score. At step 350, the processor orders users according to their risk score. Where steps 310, 320, and 330 are carried out for a plurality of users, each wearing an input device, at step 340 a risk score can be generated for each user. The users may then be placed in a list and ordered according to their risk score at step 350. In one example, the users may be arranged in descending order, where the users with the highest risk scores are placed at the top of the list and the users with the lowest risk scores are placed at the bottom of the list. Alternatively, the users may be arranged in ascending order. Ordering the users according to risk score may serve to triage users requiring a clinical assessment. Users with the highest risk scores, ordered in the list, may be booked for clinicals assessment sooner than those with lower risk scores.

In some cases, the risk score for more than one user in the list of users may be the same. The processor may recognize when two or more users have the same risk score and initiate a tie breaking method. The tie breaking method may include calculating a tie breaking score. The tie breaking score may be determined using a ranked list of risk score contributors. For example, where the risk factor and the alert total are used to calculate the risk score, the risk factor may be ranked higher than the alert total. In an example case, a first user may have a risk factor of 2 and an alert total of 3 and a second user may have a risk factor of 3 and an alert total of 2, but both users have a risk score of 5. The risk factor may then be used as the tie breaking score, as it is ranked higher than the alert total. Therefore, the second user would be placed ahead of the first user in the list. Alternatively, the alert total may be ranked higher than the risk factor and used as the tie breaking score. The tie breaking score may also be calculated using some combination of the contributors used in the risk score equation and/or data not used in the risk score equation.

As another example, the tie breaking score may be determined using one or more of the aforementioned additional contributors to the risk score equation or model. For example, the tie breaking score may be determined based on alert patterns, foot examination data, scan data, or historical user data. For example, for three users with the same risk score, historical ulceration data may be used as the tie breaking score.

As a third example, the tie breaking score may also be determined using any additional equation or model. The additional equation or model may be different than the risk score equation or model. In one example, the risk score may be calculated by a weighted sum of an alert total from a first information type, an alert total for a second information type, a risk factor for a first information type, and a risk factor for a second information type. The tie breaking score may be a sum of the alert totals for the first information type and the second information type. Rather than a single tie breaking score, a ranked list of tie breaking scores may be used to address a situation where a first tie breaking score is equal for two or more users. The list of tie breaking scores may be ranked by most important tie breaking score to least important tie breaking score. The processor may recognize when the first tie breaking score is equal for two or more users and proceed to the second tie breaking score in the list.

At step 360, a timeline may optionally be generated for each user based on the risk score, and the timeline can optionally be presented to the user or to a clinician. The timeline can be a care timeline. High risk scores may be associated with urgent care timelines, and low risk scores may be associated with non-urgent care timelines. For example, users whose risk scores fall within a low range of risk scores may have a care timeline generated that recommends a clinical assessment within 6 months, but users whose risk scores fall within a high range may have a care timeline generated that recommends a clinical assessment within 2 weeks.

Method 300 may be repeated regularly, as it is expected that users' conditions and the information alerts generated for each user will change over time. For example, method 300 may be repeated monthly. A user with a low risk score for a first execution of method 300 may have a higher risk score for a second execution of method 300, and will be ordered higher up on the list of users at step 350 during the second execution.

Referring now to FIG. 4, shown therein is a method of generating a risk factor. Method 400 may be carried out by input device 100.

At step 410, a plurality of sensor readings is collected for a user. The plurality of sensor readings may be collected using an input device which includes one or more sensors and/or sensor types. For example, where the method is carried out by input device 100, the input device 100 is provided as insole 101. Other non-wearable input devices that may collect a plurality of sensor readings from a user include sensorized mats, sensorized platforms, sensorized treadmills, sensorized equipment, etc. The data collected at 410 may include time-series sensor readings collected with the sensors included in the input device. For example, for the insole 101, the plurality of sensor readings collected from the user may include pressure readings, temperature readings, and accelerometer readings. The plurality of sensor readings collected with the input device may be received by a processor.

At step 420, one or more comparison metrics are calculated based on the plurality of sensor readings collected at step 410. The one or more comparison metrics are metrics derived from the plurality of sensor readings, which can be compared to a threshold at step 430. Calculating the one or more comparison metrics may include post-processing the collected data (e.g. applying filters and removing noise) and applying models or equations to the collected data.

At step 430, the one or more comparison metrics is compared to a threshold, to determine if a threshold requirement is satisfied. The one or more comparison metrics may be compared to the threshold over a specified time period (e.g. one day, one month, etc.). The threshold is a value that indicates a change in ulceration risk. Comparison metrics that fall on one side of the threshold (either above or below the threshold, depending on the type of sensor readings collected) are considered “safe”. In this case, the threshold requirement is satisfied, and method 400 does not proceed any further. The system may return to step 410 and repeat method 400 from the beginning. Optionally, the system may generate a risk factor, where the generated risk factor is a baseline value (e.g. zero). Comparison metrics that fall on the other side of the threshold (the opposite side as that mentioned above) are considered “unsafe”, and the risk of ulceration is increased. In this case, the threshold requirement is not satisfied, and the method proceeds to step 440.

At step 440 an information alert is generated. An information alert is an alert that indicates that the threshold requirement was not satisfied at step 430, and optionally presents relevant data pertaining to the threshold requirement and why it might not have been satisfied. Relevant data may include the one or more comparison metrics, the collected sensor readings, anatomical locations of the user associated with the comparison metrics where the threshold requirement was not satisfied, additional data such as activity data and/or historical user data which may provide context for why the threshold was not satisfied, etc. The activity data may be generated by the same input device or a separate input device. The activity data may be generated by an IMU, for example, and may include step count, heart rate, or other indicators of an activity being performed and its intensity. The information alert may be generated by a processor, such as processor 120 or the remote processor. The information alert may additionally be provided visually on an output device of the system, such as a display, or it may be provided audibly or haptically. For example, the information alert may be provided in the form of a pop-up notification on the display. In other examples, information alerts may be passed directly to a remote processor and the display may be accessible by a healthcare practitioner or remote patient monitor.

At step 450, a risk factor is generated. The risk factor may be a risk factor qualitative level, such as “low”, “medium”, “medium-high”, or “high”, or the risk factor may be a risk factor quantitative level, such as a number from 1 to 4. The generated risk factor depends on the type of information alert generated at step 440 (and hence the type of sensor readings collected at 410).

At step 460, an action alert is generated. An action alert is an alert which indicates an action that may be taken to decrease ulceration risk, such that when method 400 is repeated, the one or more comparison metrics will satisfy the threshold requirement at step 430. The action alert that is generated may depend on the type of information alert that is generated, which depends on the type of sensor readings that are collected by the input device. The action alert may be provided visually on an output device of the system, such as a display, or it may be provided audibly or haptically. For example, the action alert may be provided in the form of a pop-up notification on the display.

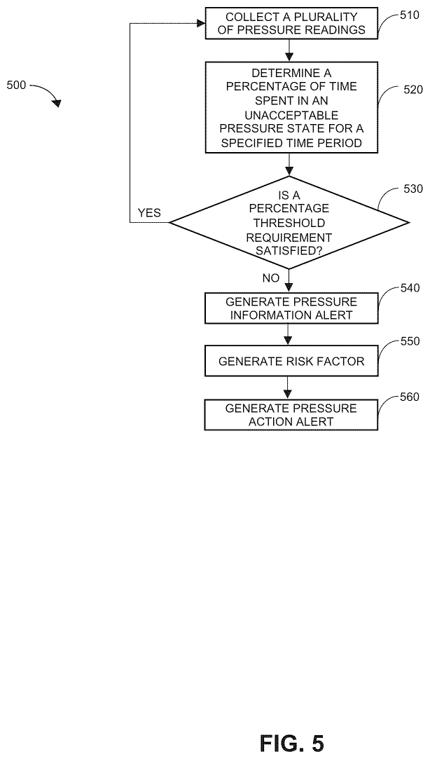

Referring now to FIG. 5, shown therein is an example method 500, which applies the method 400 of FIG. 4 to a plurality of pressure readings. The example method 500 identifies situations when a user's plantar pressures exceed a safe threshold. If the user's plantar pressures are maintained above the threshold, inflammation and ulceration may occur. Alerts are generated to support users in reducing their plantar pressures in response to these situations. Example method 500 may be carried out by input device 100.

At step 510, a plurality of pressure readings are collected using the pressure sensors 116 of input device 100 over a specified time period. The plurality of pressure readings may be collected as time-series data and may be associated with different plantar locations on a user's foot.

At step 520, a percentage of time spent in an unacceptable pressure state is determined for the specified time period (i.e. the one or more comparison metrics determined at step 420 of method 400). An unacceptable pressure state occurs when at least one pressure sensor, of the one or more pressure sensors of the input device 100, produces a pressure reading that exceeds a pressure threshold. The pressure threshold may be 50 mmHg. The pressure threshold may be a pressure magnitude that when exerted on or by the plantar surface for long durations increases an individual's risk for foot inflammation and ulceration. The percentage of time spent in an unacceptable state may be determined by summing the time periods when an unacceptable pressure state is identified within the specified time period, and dividing the sum by the usage time of the device in the specified time period. The usage time of the device may be determined by summing time periods within the specified time period when the sensor readings from any of the sensors in the input device (e.g. pressure sensors, temperature sensors, etc.) contain non-baseline values. The specified time period may be at least one day.

At step 530, the percentage of time spent in an unacceptable pressure state for the specified time period is compared to a percentage threshold to determine whether a percentage threshold requirement is satisfied. The percentage threshold may be in a range of 40% to 50%. In this example, the percentage threshold requirement is satisfied when the percentage of time spent in an unacceptable pressure state is less than the percentage threshold. If the percentage threshold requirement is satisfied at step 530, example method 500 does not proceed any further, and the system returns to step 510 to continue collecting pressure readings. Optionally, the system may generate a risk factor with a baseline value (e.g. zero), since there is no risk associated with the pressure readings. Alternatively, if the percentage threshold requirement is not satisfied (i.e. the percentage of time spent in an unacceptable pressure state exceeds the percentage threshold), the method proceeds to step 540.

At step 540 a pressure information alert is generated. The pressure information alert may indicate that the percentage threshold requirement was not satisfied. The pressure information alert may also provide the pressure readings for the specified time period, the plantar locations associated with the pressure readings, the percentage of time spent in an unacceptable pressure state for the specified time period, activity data for the specified time period, and/or historical user data of the user. The pressure information alert may be presented visually on an output device of the system, such as a display, or it may be provided audibly or haptically. For example, the pressure information alert may be provided in the form of a pop-up notification on the display, notifying the user that their plantar pressures are high.

At step 550, a risk factor is generated. In this example, where the method is applied to a plurality of pressure readings and the information alert that is generated is a pressure information alert, the generated risk factor may be a “low” risk level.

At 560, a pressure action alert may be generated. A pressure action alert is an alert which indicates an action that may be taken to offload plantar pressures. If the action is taken and method 500 is repeated, at step 530, the percentage of time spent in an unacceptable pressure state will in theory be less than the percentage threshold, and the percentage threshold requirement will be satisfied. The pressure action alert may be presented visually on an output device of the system, such as a display, or it may be provided audibly or haptically. For example, the pressure action alert may be provided in the form of a pop-up notification on the display.

The pressure action alert may include a pressure offloading alert. The pressure offloading alert may include an automated, self-directed pressure offloading procedure.

The procedure may be presented on an output device of the system, and the user may follow the steps of the procedure to safely reduce pressure on their feet.

The pressure action alert may include an activity modification alert. The activity modification alert may be presented in the form of a step count goal (e.g. as described in method 800, below). The step count goal may encourage users to gradually increase their activity level, to achieve the cardiovascular, metabolic, and mental health benefits of engaging in physical activity, while discouraging users from abruptly increasing activity levels in ways that might be unsafe. Alternatively, the activity modification alert may be presented in the form of a modified weight bearing activity alert. The modified weight bearing activity alert may include an automated, self-directed procedure for performing a modified weight bearing activity. The modified weight bearing activity alert may encourage users to continue to engage with physical activities, but in ways that minimize pressures on their feet to reduce ulceration risk.

The pressure action alert may include a foot examination alert. The foot examination alert may recommend that a user perform an inspection of their feet for abnormalities (e.g. visible abnormalities), abnormal colors, calluses, foot deformities, and temperature differences that can be determined by touch.

The pressure action alert may include a custom orthotic alert. The custom orthotic alert may recommend an orthotic to the user that if worn, will assist the user in offloading pressures underfoot. The custom orthotic alert may provide a recommended orthotic, where the recommended orthotic is one or more of a single dancer pad, a double dancer pad, a metatarsal bar, a metatarsal pad, a u-shaped metatarsal pad, an extrinsic post, and/or a heel lift, as well as a recommended placement for the orthotic. The recommended orthotic and the recommended placement for the orthotic may be determined based on the plantar locations associated with pressure readings determined to exceed the pressure threshold of 50 mmHg at step 520. In particular, if pressure readings exceed the pressure threshold at the second metatarsal of a user's foot, the custom orthotic alert may recommend a metatarsal pad or a u-shaped metatarsal pad to the user. If pressure readings exceed the pressure threshold at one or more of the metatarsal heads of a user's foot, the custom orthotic alert may recommend a single dancer pad or double dancer pads to the user.

The pressure action alert may include a surgical alert. The surgical alert may recommend a surgery to the user that if performed, may prevent ulceration. The surgical alert may be generated based on the pressure readings collected for the user. In particular, an increase in the pressure readings at a user's midfoot may indicate a collapsed arch. The surgical alert may present a recommendation for a surgical relief cut in response to the increased pressures, to help relieve pain from the collapsed arch and restore function to the arch. In one example, the surgical alert recommending a relief cut is generated if pressure readings at the midfoot of a user are determined to exceed the 50 mmHg pressure threshold at step 520.

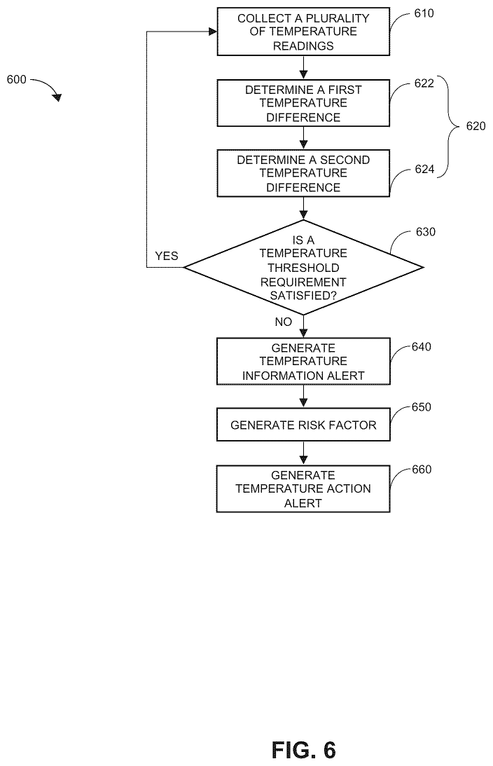

Referring now to FIG. 6, shown therein is an example method 600, which applies the method 400 of FIG. 4 to temperature readings. The example method 600 identifies situations when a user's plantar temperatures exceed a safe threshold, which may indicate that inflammation and tissue damage are occurring. Alerts are generated to support users in reducing their plantar temperatures in response to these situations. Example method 600 may be carried out by input device 100.

At step 610, a plurality of temperature readings are collected over a time period using the temperature sensors 110 of input device 100. The plurality of temperature readings may be collected as time-series data and may be associated with different plantar locations on a user's foot.

At step 620, which includes steps 622 and 624, a first temperature difference and a second temperature difference are determined (i.e. the one or more comparison metrics determined at step 420 of method 400). At 622, the first temperature difference is determined for a first time by comparing a first temperature reading at a first plantar location to a first temperature reading at a second plantar location, and taking the difference between the two readings. At 624, the second temperature difference is determined for a second time by comparing a second temperature reading at the first plantar location to a second temperature reading at the second plantar location, and taking the difference between the two readings. The time period includes the first time and the second time. The first location and the second location may be on opposing feet on a user, in which case the first temperature difference and the second temperature difference are contralateral temperature differences. Alternatively, the first location and the second location may be on a same foot of a user, in which case the first temperature difference and the second temperature difference are ipsilateral temperature differences. For example, the first location may be the hallux of a user's right foot, and the second location may be the third metatarsal of the user's right foot. The ipsilateral temperature difference may also be taken by comparing a temperature reading at a first plantar location to an average of all temperature readings for that foot for the first time or second time. For example, the first location may be the hallux of a user's right foot, and the second location may be the user's entire foot (with an averaged temperature reading). The first time and the second time may be consecutive, and they may be one hour apart.

At step 630, the first temperature difference and the second temperature difference are compared to a temperature threshold, to determine if a temperature threshold requirement is satisfied. The temperature threshold may be a skin temperature that is associated with inflammation and pre-ulceration. The temperature threshold may be at least 2.0° C. The temperature threshold may be 2.2° C. (4° F.). In this example, the temperature threshold requirement is satisfied if either one or both of the first temperature difference and the second temperature difference are below the temperature threshold. If the temperature threshold requirement is satisfied at step 640, example method 600 does not proceed any further, and the system returns to step 610 to continue collecting temperature readings. Optionally, the system may generate a risk factor, where the generated risk factor is a baseline value (e.g. zero), since there is no risk associated with the temperature readings. Alternatively, the temperature threshold requirement is not satisfied if both the first temperature difference and the second temperature difference exceed the temperature threshold.

If the temperature threshold requirement is not satisfied at step 630, at step 640 a temperature information alert is generated. The temperature information alert may indicate that the temperature threshold requirement was not satisfied. The temperature information alert may also provide the plantar temperature readings for the first and second times, the plantar locations associated with the plantar temperature readings, the first and second temperature differences, activity data for a time period around the first and second time instants, and/or historical user data of the user. The temperature information alert may be presented visually on an output device of the system, such as a display, or it may be provided audibly or haptically. For example, the temperature information alert may be provided in the form of a pop-up notification on the display, notifying the user that their plantar temperatures are high.

At step 650, a risk factor is generated. In this example, where the method is applied to a plurality of temperature readings and the information alert that is generated is a temperature information alert, the generated risk factor may be a “medium” risk level.

At step 660, a temperature action alert may be generated. A temperature action alert is an alert which indicates an action that may be taken to reduce plantar temperatures and thereby inflammation. If the action is taken and method 600 is repeated, at step 630, either one or both of the first temperature difference and the second temperature difference will in theory be less than the temperature threshold. The temperature action alert may be presented visually on an output device of the system, such as a display, or it may be provided audibly or haptically. For example, the temperature action alert may be provided in the form of a pop-up notification on the display.

The temperature action alert may include a pressure offloading alert. The pressure offloading alert may include an automated, self-directed pressure offloading procedure. The procedure may be presented on an output device of the system, and the user may follow the steps of the procedure to safely reduce pressure on their feet. By reducing pressure on their feet, inflammation may decrease to prevent the formation of ulcers.

The temperature action alert may include an activity modification alert. The activity modification alert may be presented in the form of a step count goal. The step count goal may encourage users to gradually increase their activity level, to achieve the cardiovascular, metabolic, and mental health benefits of engaging in physical activity, while discouraging users from abruptly increasing activity levels in ways that might be unsafe. Alternatively, the activity modification alert may be presented in the form of a modified weight bearing activity alert. The modified weight bearing activity alert may include an automated, self-directed procedure for performing a modified weight bearing activity. The modified weight bearing activity alert may encourage users to continue to engage with physical activities, but in ways that minimize pressures on their feet to reduce inflammation and ulceration risk.

The temperature action alert may include a vascular assessment requirement alert, which recommends a vascular assessment to the user. The vascular assessment requirement alert may recommend a vascular assessment including an ankle brachial index test with segmental pressures and a doppler waveform analysis.

The temperature action alert may include a foot examination alert. The foot examination alert may recommend that a user perform an inspection of their feet for abnormalities (e.g. visible abnormalities), abnormal colors, calluses, foot deformities, and temperature differences that can be determined by touch.

The temperature action alert may include a custom orthotic alert. The custom orthotic alert may recommend an orthotic to the user that if worn, will assist the user in offloading pressures underfoot. The custom orthotic alert may provide a recommended orthotic, where the recommended orthotic is one or more of a single dancer pad, a double dancer pad, a metatarsal bar, a metatarsal pad, a u-shaped metatarsal pad, an extrinsic post, and/or a heel lift, as well as a recommended placement for the orthotic. The recommended orthotic and the recommended placement for the orthotic may be determined based on the first and second plantar locations associated with the first and second temperature differences that are determined to exceed the temperature threshold at step 630. In particular, if the first temperature difference and the second temperature difference exceed the temperature threshold, and the first or second plantar location is the second metatarsal of a user's foot, the custom orthotic alert may recommend a metatarsal pad or a u-shaped metatarsal pad to the user. Alternatively, if the first temperature difference and the second temperature difference exceed the temperature threshold, and the first or second plantar location is a metatarsal head of a user's foot, the custom orthotic alert may recommend a single dancer pad or double dancer pads to the user. Increased temperatures may be a result of high pressures at these plantar locations, and the recommended orthotics may help to offload pressures in order to decrease the temperatures at these locations.

Optionally, at step 660, foot examination data may be received at the processor. The foot examination data may indicate a presence or an absence of an abnormality on a foot of the user. The abnormality may be a visual abnormality. The foot examination data may also include a presence or absence of abnormal colors, calluses, foot deformities, and temperature differences that can be determined by touch. The temperature action alert may only be generated if the foot examination data indicates a presence of an abnormality.

Method 600 may include optional steps for filtering the plurality of temperature readings or clearing the temperature information alerts and/or the temperature action alerts. These optional steps may be taken to eliminate “false positive” alerts, which are temperature alerts that are generated that are not actually a result of inflammation/ulceration of the foot.

In one example, method 600 includes a filtering procedure, which occurs prior to determining the first temperature difference and the second temperature difference at steps 622 and 624. The first step in the filtering procedure is to collect activity data over a time period that includes the first time and the second time. The activity data may be collected using the same input device that collects the plurality of temperature readings, such as the insole 101. Alternatively, the activity data may be collected using a separate input device. For example, the activity data may be collected using a wrist-worn accelerometer in communication with the input device 100. The activity data may include accelerometer data, gyroscope data, and/or pressure data. The next step in the filtering procedure is to identify an occurrence of an activity based on the activity data. The occurrence of an activity may be determined by inputting the activity data into an activity classification algorithm. An example of an activity classification algorithm that may be used to classify sensor data is described in U.S. Pat. No. 11,526,749 entitled “METHOD AND SYSTEM FOR ACTIVITY CLASSIFICATION”, the entirety of which is incorporated herein by reference. The activities that may be identified using the activity classification algorithm include donning the input device 100, doffing the input device 100, and high intensity activities (e.g. walking, running, jumping, etc.). The last step in the filtering procedure is to filter out changes in the plurality of temperature readings that temporally align with the occurrence of the activity or a set amount of temperature readings surrounding the occurrence of the activity. Changes in the plurality of temperature readings may be filtered out if they are sudden (i.e. if a rate of change in the temperature readings exceeds a rate of change threshold). For example, when a user dons the insoles, the temperature sensors in the insoles are expected to detect a rapid increase in temperature, from a temperature baseline (typically room temperature), to the user's skin temperature. If the insoles are donned separately (e.g. if the left insole is donned first, then the right insole donned second), one insole may measure temperature readings at skin temperature, and the other insole may still measure temperature readings at the temperature baseline at a point in time. In this case, the first and second temperature differences may exceed the temperature threshold, which would trigger the generation of a temperature information alert, however the exceedance is a result of donning the insole, not as a result of inflammation/ulceration. Therefore, the plurality of temperature readings that temporally align with the activity of donning the insole 101 can be filtered out, such that a temperature information alert is not generated at step 640.

In another example, method 600 includes a procedure for filtering the plurality of temperature readings based on historical user data. Historical user data may include a history of a condition that causes chronic limb temperature differences. For example, the condition may be peripheral arterial disease, macrovascular disease, a stroke, an asymmetric neurological disease (e.g. impaired sympathetic tone), muscle mass asymmetry, or edema. The processor may receive the historical user data, identify chronic temperature differences, and normalize the plurality of temperature readings to eliminate the chronic temperature differences. Filtering out these chronic temperature differences will prevent a false temperature information alert from being generated at step 640.

Both the aforementioned examples may be applied as filtering steps, for filtering the temperature readings to prevent the generation of a temperature information alert at step 640 and a temperature action alert at step 660. Alternatively, the examples may be applied after the alerts have been generated, to establish criteria for clearing the already-generated alerts.

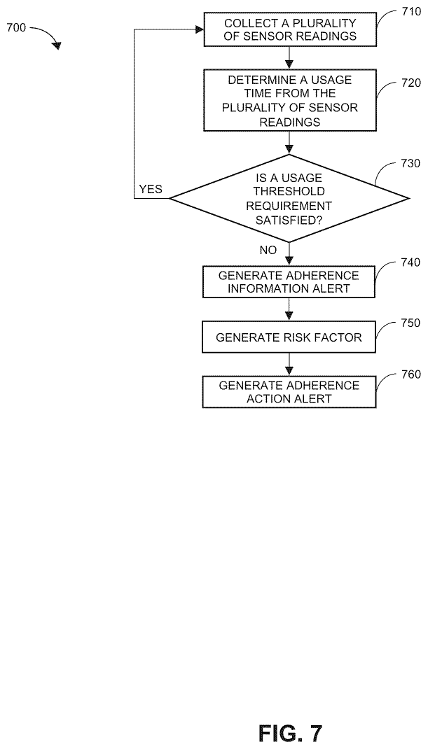

Referring now to FIG. 7, shown therein is an example method 700, which applies the method 400 of FIG. 4 to a plurality of sensor readings. The example method 700 identifies instances when a user has low adherence to the system (i.e. when their usage time is below a usage threshold) and generates alerts to encourage the user to increase their adherence. Not adhering to good usage practices (wearing the input device regularly, charging the input device at recommended intervals, etc.) can create unsafe conditions for the user, as the formation of ulcers may go undetected. By the time the ulcers are detected, more complex interventions may be required. Example method 700 may be carried out by input device 100.

At step 710, a plurality of sensor readings are collected. The plurality of sensor readings may be collected by any sensors included in the input device. For example, for the insole 101, the plurality of sensor readings may be a plurality of pressure readings generated by the pressure sensors 116, a plurality of temperature readings generated by the temperature sensors 110, and a plurality of accelerometer readings generated by IMU 122. The plurality of sensors readings may be collected as time-series data.

At step 720, a usage time may be determined from the plurality of sensor readings for a specified time period (i.e. the one or more comparison metrics determined at step 420 of method 400). The usage time may be determined by analyzing the time-series sensor readings to identify and sum time periods within the specified time period when the sensor readings contain non-baseline values. The specified time period may be one day. For example, where both the pressure sensors 116 and the temperature sensors 110 of the input device 100 record non-baseline values between 10:30 am and 2:15 pm in one day (e.g. when the user is out running errands), and the temperature sensors 110 of input device 100 record non-baseline values between 2:15 μm and 3:30 pm for the same day (e.g. while the user is sitting down), the usage time for that day would be 5 hours. To differentiate baseline versus non-baseline values, a threshold may be established to determine when a sensor reading is no longer a baseline value. The threshold may include a single sensor type threshold or may include multiple sensor type thresholds. One example of multiple sensor type thresholds may include a temperature threshold of 24° C., pressure threshold of 30 mmHg, and a step count greater than one step. In the example, all or a percentage of the multiple thresholds must be exceeded for the sensor readings to be counted as non-baseline values.

At step 730, the usage time is compared to a usage threshold, to determine whether a usage threshold requirement is satisfied. The usage threshold may be a usage time of the input device that is shown to decrease risk of ulceration. The usage threshold may be 4.5 hours per day. In this example, for the usage threshold requirement to be satisfied, the daily usage time must exceed 4.5 hours. In the above example, where the usage time was determined to be 5 hours, the usage threshold requirement would be satisfied, as 5 hours is greater than 4.5 hours. If the usage threshold requirement is satisfied at step 730, example method 700 does not proceed any further, and the system returns to step 710 to continue collecting sensor readings. Optionally, the system may generate a risk factor, where the generated risk factor is a baseline value (e.g. zero), since there is no risk associated with the usage time. Alternatively, the usage threshold requirement is not satisfied if the daily usage time is less than 4.5 hours.

If the usage threshold requirement is not satisfied at step 730, at step 740 an adherence information alert is generated. The adherence information alert may provide an indication that the usage threshold requirement was not satisfied. The adherence information alert may also provide the usage time, trends in the usage time over a longer period, activity data for the usage time, and/or historical user data of the user. The adherence information alert may be presented visually on an output device of the system, such as a display, or it may be provided audibly or haptically. For example, the usage information alert may be provided in the form of a pop-up notification on the display, notifying the user that their usage time is low.

At step 750, a risk factor is generated. In this example, where the method is applied to a usage time and the information alert that is generated is an adherence information alert, the generated risk factor may be a “medium-high” risk level or “high” risk level.

At step 760, an adherence action alert may be generated. An adherence action alert is an alert which indicates an action that may be taken to improve usage of the input device, and thereby decrease ulceration risk. If the action is taken and method 700 is repeated, at step 730, the usage time will in theory be greater than the usage threshold of 4.5 hours. The adherence action alert may be presented visually on an output device of the system, such as a display, or it may be provided audibly or haptically. For example, the adherence action alert may be provided in the form of a pop-up notification on the display.

The adherence action alert may include an engagement incentivization alert. The engagement incentivization alert may gamify or provide incentives for a user to increase their engagement with the input device and/or the processor/display. An example of a method that may be used to promote increased engagement of an input device is described in US Patent Application Publication No. 2023/0309933 entitled “SYSTEM AND METHOD FOR ANALYZING USE OF A WEARABLE DEVICE”, which is incorporated herein by reference.

The adherence action alert may include a foot examination alert. The foot examination alert may recommend that a user perform an inspection of their feet for abnormalities (e.g. visible abnormalities), abnormal colors, calluses, foot deformities, and temperature differences that can be determined by touch.

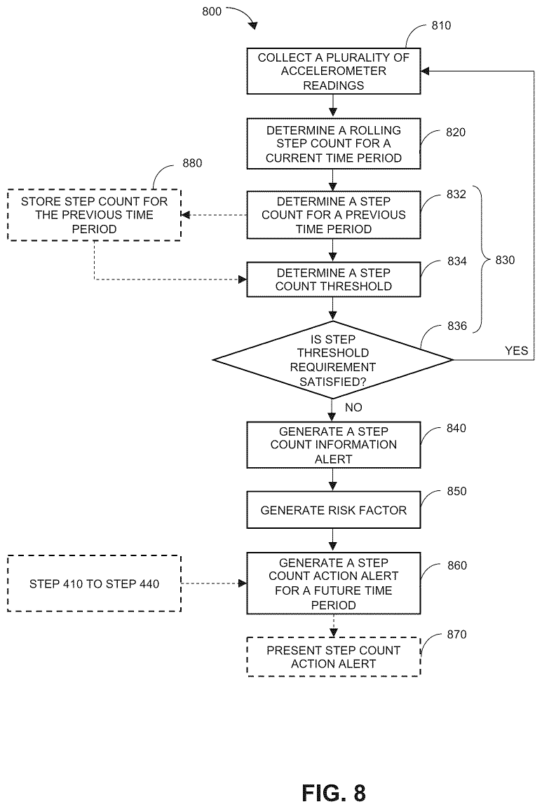

Referring now to FIG. 8, shown therein is an example method 800, which applies the method 400 of FIG. 4 to accelerometer readings. Example method 800 shows a method for identifying abrupt increases in activity for a user, and for generating an activity level for the user to prevent injury and other safety issues that may otherwise arise from the abrupt increase in activity. Example method 800 may be carried out by input device 100.

Method 800 may benefit users with unpredictable activity levels, which may lead to injury or other unsafe conditions. For example, for a user with diabetic peripheral neuropathy, large increases or decreases in activity may increase risk of ulceration. A user with abrupt increases in activity (e.g. weight bearing activities, gait activities, etc.) may experience higher than normal pressure levels in the feet, which can result in diabetic foot ulcers. A user with low activity levels or consistent decreases in activity level may not experience the cardiovascular, metabolic, and mental health benefits resulting from increased mobility in populations at high risk of developing diabetes health complications (“high-risk populations”), such as diabetic foot ulcers, heart disease, kidney disease, oral health issues, mental health issues, etc. Method 800 may be used to generate a balanced activity level that will decrease the likelihood of high-activity related injuries, while maintaining the benefits of moderate and consistent activity in high-risk populations.

At step 810, a plurality of accelerometer readings are collected. The plurality of accelerometer readings may be collected by an IMU or an accelerometer included in the input device. For example, for the input device 100, the plurality of accelerometer readings may be generated by the IMU 122, which includes an accelerometer. The plurality of accelerometer readings may be collected as time-series data and collected over a time period. The time period may include a rolling time period that moves as accelerometer readings are collected from the input device worn by the user (e.g. a 24-hour time period that moves with the current instant) or may include a static time window that only moves when a segment of the time period is completed (e.g. after a day, week, month, year). The time period may include a current time period (e.g. at least three days) during which the user has worn the input device. If the user has not worn the input device for three consecutive days, the current period may include the last three non-consecutive days that the input device collected data or the current period may include the last three consecutive days, including days where no data was collected (e.g. step count of 0 for some of the days). The time period may also include a previous time period (e.g. one month) during which historical accelerometer readings were recorded and stored in the system's memory. The previous time period may be longer than the current time period. The previous time period may include the data preceding the current time period or may overlap with the current time period. For example, if the current time period is three days (i.e. with the third day being the current date) and the previous time period is one month, the three days of the current time period may overlap the last few days of the month (i.e. the previous time period). In the example method 800 described below, the step count is determined using an accelerometer, however, it is appreciated that other sensors and input devices may be used to measure step count. For example, step count may be determined using the pressure sensor array 114 in the insole 101, where a pressure pattern is detected to indicate each step taken by the user.

At step 820, a rolling step count (i.e. the one or more comparison metrics) is determined based on the plurality of accelerometer readings collected at step 810. The processor 120 or remote processor may determine a step count using a step count algorithm. The step count algorithm may determine the number of steps taken by the user from the plurality of accelerometer readings using a high and a low threshold. A step may be counted when the accelerometer readings start below the low threshold, exceed the high threshold, and then retreat below the low threshold. The rolling step count is determined for the current time period, using the plurality of accelerometer readings collected during the current time period. The rolling step count is a step count that increases as the user completes a step. The rolling step count may extend over a designated period of time, for example, one day. The rolling step count may increase throughout the designated period of time until the end of the designated period of time, at which point, the rolling step count may cease. For example, the rolling step count may increase as the user completes steps from 12:01 AM in the morning until midnight of the same day. Once midnight has passed, the rolling step count is reset to zero.

At step 830, which includes steps 832, 834, and 836, it is determined if a step threshold requirement is satisfied. The step threshold requirement is satisfied when the rolling step count is less than a step count threshold. The step count threshold may be a set value (e.g. 10,000 steps), or the step count threshold may be a value that is relative to the user and their previous data.

To determine a step count threshold that is relative to the user and their data, a step count is first determined for the previous time period at step 832. At step 832, the plurality of accelerometer readings from the previous time period are converted into a step count. This may be done using the step count algorithm example mentioned above.

At step 834, a step count threshold is determined from the step count for the previous time period determined at step 832. The step count threshold is determined as a percentage of a step count baseline. For example, the step count threshold may be 50% of a step count baseline.

The step count baseline may be the step count for the previous time period, determined at step 832. For example, if a user recorded 130,000 steps in the previous month, the step count baseline would be 130,000 steps. If the step count threshold is 50% of the step count baseline, the step count threshold would be 65,000 steps. Alternatively, the step count baseline may be determined based on step counts for a plurality of previous time periods. The step counts for the previous time periods may be stored in the memory of the system, as indicated in optional step 880. The step counts for the previous time periods may be recalled from the memory to calculate the step count baseline. The step count baseline may be determined using any equation that combines the step counts for the plurality of previous time periods. For example, the step count baseline may be calculated as an average step count for the previous time periods. In one example, a plurality of previous time periods includes three previous time periods, each previous time period one month in length (e.g. the calendar month prior to the current date, and the two preceding months). The step count for each month is determined and summed to create a total step count of 450,000 steps for the three months. The step count baseline is determined to be the average step count for the plurality of time periods, which is 150,000 steps (450,000 steps divided by 3). The step count threshold is then determined to be 50% of the step count baseline, which is 75,000 steps.

At step 836, the rolling step count is compared to the step count threshold, to determine if a step threshold requirement is satisfied. The step count threshold indicates an activity level that may be considered unsafe for the user if exceeded in a short span of time, such as the current time period. The step threshold requirement is satisfied when the rolling step count is less than the step count threshold. In this case, the activity level is considered “safe”. When the step threshold requirement is satisfied, method 800 does not proceed any further, and instead, the system may return to step 810 and repeat method 800 from the beginning. Optionally, the system may generate a risk factor, where the generated risk factor is a baseline value (e.g. zero), since there is minimal risk associated with the step count. When the rolling step count does not satisfy the step threshold requirement (i.e. the rolling step count exceeds the step count threshold), the activity level is considered “unsafe”, and the user's risk of ulceration increases. When the step threshold requirement is not satisfied, the method proceeds to step 840.

At step 840, a step count information alert is generated. The step count information alert is an alert that indicates that the step threshold requirement was not satisfied at step 830, and optionally presents relevant data pertaining to the step threshold requirement. Relevant data may include the rolling step count, the step count threshold, the step count baseline, high pressure or temperature zones on the feet, methodology for generating the step count information alert, etc. The step count information alert may be generated by a processor, such as processor 120 or the remote processor. The information alert may additionally be provided visually on an output device of the system, such as a display, or it may be provided audibly or haptically. For example, the step count information alert may be provided in the form of a pop-up notification on the display, notifying the user that their activity levels for the day are high.

At step 850, a risk factor is generated. In this example, where the method is applied to a plurality of accelerometer readings and the information alert that is generated is a step count information alert, the generated risk factor may be a “low” risk level.

At step 860, a step count action alert is generated for a future time period. The step count action alert is an alert which indicates an action that may be taken to decrease ulceration risk, such that when method 800 is repeated, the one or more comparison metrics will satisfy the step threshold requirement at step 830. The step count action alert may include a step count goal for a future time period, which is a “safe” activity level generated for the user. The future time period may be the same length of time as the previous time period. The future time period (e.g. one month) may be consecutive with the current time period, meaning the future time period may start directly after the current time period (e.g. the next minute, hour, day, etc.). Alternatively, the future time period may start when the wearable device is next worn by the user or at some other non-consecutive time. The step count goal may be generated with a step count goal equation that uses the step count data from the previous time period. The step count goal equation may be stored in and carried out by the processor 120 or the remote processor. For example, the step count goal may be determined as at least a 10% increase from the step count for the previous time period determined at step 832. The step count goal for the future time period may be broken down into divisions of steps for smaller periods in the future time period. For example, the future time period may be one month (i.e. 30 days), and the step count goal may be calculated as 180,000 steps. The step count goal may then be broken down to 6,000 steps for each day to help the user stay on track. The daily step count goal may then be updated and altered depending on rolling step count for each day in the month.

The step count action alert may include a modified weight bearing activity alert. The modified weight bearing activity alert may include an automated, self-directed procedure for performing a modified weight bearing activity. The modified weight bearing activity alert may encourage users to continue to engage with physical activities, but in ways that minimize pressures on their feet to reduce ulceration risk.

At optional step 870, the step count action alert is presented to the user. Presenting the step count action alert may include presenting the step count goal (calculated at step 860) to the user. The step count goal may be presented to the user to encourage a reduced activity level over the future time period. Presenting the step count action alert directly to the user allows the user to self-direct their own care. The step count action alert may additionally be provided visually on an output device of the system, such as a display, or it may be provided audibly or haptically. For example, the step count action alert may be provided in the form of a pop-up notification on the display. In some examples, the step count action alert may be presented by displaying the step count goal alongside the rolling step count for the current period. Presenting both allows the user to compare the values and create a plan for achieving, and not exceeding the step goal.

In some examples, the step count action alert is generated solely based on steps 810 to 860 of method 800, using the plurality of accelerometer readings, as described above. In other examples, the step count action alert is only generated after the method additionally proceeds through steps 410 to 440 of method 400, which uses a plurality of additional sensor readings to generate an additional information alert. A plurality of additional sensor readings may be collected over an additional time period using additional sensors on the input device (step 410). The additional time period may be the same as the current time period or some portion of the current time period. For example, if the current time period is three days, the additional time period may be one day. The plurality of additional sensor readings may be used to calculate one or more additional comparison metrics (step 420), which can be used to determine if an additional threshold requirement has been satisfied (step 430). If the additional threshold requirement is not satisfied, an additional information alert may be generated (step 440). Optionally, an additional action alert may be generated (step 460). The step count goal may then be generated and presented to the user (steps 860 and 870). In some examples, where the additional sensor readings are pressure readings, steps 410 to 440 may be carried out as steps 510 to 540 of example method 500. The additional information alert that is generated at 440 will be a pressure information alert. In another example, the additional sensor readings may be temperature readings, and steps 410 to 440 may be carried out as steps 610 to 640 of example method 600. The additional information alert generated at 440 will be a temperature information alert. Failure to satisfy the percentage threshold requirement at step 530 of example method 500 or a failure to satisfy the temperature threshold requirement at step 630 of example method 600 may be partially attributable to an abrupt increase in activity. For example, if a user experiencing diabetic peripheral neuropathy abruptly increases their activity levels, the high activity levels can cause high pressure zones on the user's feet. The high-pressure zones can result in inflammation and tissue damage in the feet, resulting in high temperature zones on the user's feet. To prevent high pressure zones and/or high temperature zones from forming as a result of the elevated activity levels, the step count action alert may be presented to the user. In another example, steps 410 to 440 may be carried out as steps 710 to 740 of example method 700, and the additional information alert generated at 740 will be an adherence alert. Failure to satisfy the usage threshold requirement at step 730 of method 700 may indicate a lack of activity for the user. As noted above, there are several benefits to moderated increases in activity for users with diabetes. The step count goal may be generated for a user who has not satisfied the usage threshold requirement. The presentation of the step count goal may remind the user to increase usage time of the insole 101 by increasing their step count.

A step count check may also be completed to evaluate the user's compliance with the generated activity level (e.g. the step count goal). When the future time period begins, a plurality of accelerometer readings may be collected over the future time period. The future time period may begin when the current time period is equivalent to the first day of the future time period. To evaluate the user's compliance with the step count goal for the future time period, the step count goal may require normalization to the length of the current time period. For example, if the future time period is one month (i.e. 30 days), the step count goal is 150,000 steps, and the current time period is 3 days, the normalized step count goal may be 15,000 steps for 3 days. A step count for the future time period may be generated based on the plurality of accelerometer readings and compared to the step count goal. If the step count for the future time period is equivalent to or greater than the step count goal, then the step count goal has been satisfied. When the step count goal is satisfied an alert may be generated and presented to the user through an output device of the system. The alert may indicate that the user has reached the step count goal and should take precautions to avoid exceeding the step count goal for the future time period.

In another example, the step count goal may be reduced by a set amount (e.g. 10%) and the step count for the future time period may be compared to the reduced step count amount. If the step count for the future time period is equivalent to or greater than the reduced step count amount, then the reduced step count amount has been satisfied. When the reduced step count amount is satisfied the alert may be generated and presented to the user or an additional user through an output device of the system. In this case, the alert is provided to the user before the step count goal is reached, which warns the user that they are close to achieving the step count goal. The user may reduce or cease activity to prevent any “unsafe” conditions that may result from exceeding the step count goal.

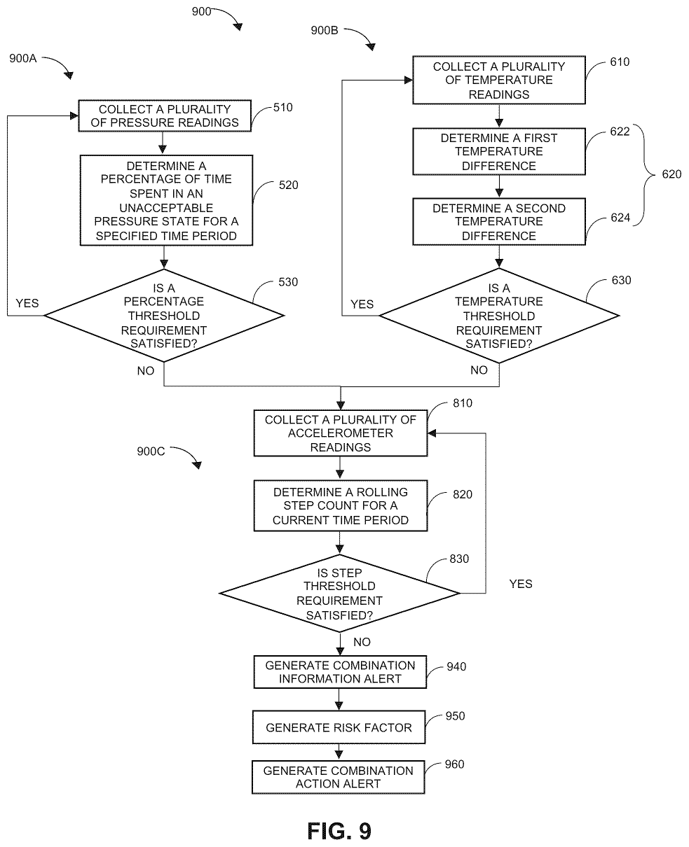

Referring now to FIG. 9, shown therein is an example method 900, which applies the method 400 of FIG. 4 to a combination of sensor reading types. Example method 900 may be carried out by input device 100.

Method 900 includes submethods 900A, 900B, and 900C. Submethods 900A, 900B, and 900C each collect a different type of sensor reading and determine whether a comparison metric calculated from the sensor readings satisfies a threshold requirement. Alerts are only generated at steps 950 and 960 if one or both of 900A and 900B do not satisfy their threshold requirements and 900C does not satisfy its threshold requirement. Method 900 may be carried out simultaneously with one or more of method 500 to 800 in FIGS. 5 to 8.

900A includes steps 510, 520, and 530 of example method 500, which applies the steps 410, 420, and 430 of method 400 to pressure readings. Steps 510 and 520 are carried out as previously described. At step 530, if the percentage threshold requirement is satisfied, submethod 900A does not proceed any further, and the system returns to step 510 to continue collecting sensor readings. If the percentage threshold requirement is not satisfied, however, the system exits submethod 900A and enters submethod 900C.

Similarly, 900B may be executed in parallel to 900A. 900B includes steps 610, 620, and 630 of example method 600, which applies the steps 410, 420, and 430 of method 400 to temperature readings. Steps 610 and 620 are carried out as previously described. At step 630, if the temperature threshold requirement is satisfied, submethod 900B does not proceed any further, and the system returns to step 610 to continue collecting sensor readings. If the temperature threshold requirement is not satisfied, however, the system exits submethod 900B and enters submethod 900C.

As can be understood from above, submethod 900C may be executed if the percentage threshold requirement is not satisfied at 530, if the temperature threshold requirement is not satisfied at 630, or if both the percentage threshold requirement and the temperature threshold requirement are not satisfied. 900C includes steps 810, 820, and 830 of example method 800, which applies the steps of method 400 to accelerometer readings. Steps 810 and 820 are carried out as previously described. At step 830, if the step threshold requirement is satisfied, submethod 900C does not proceed any further, and the system returns to step 810 to continue collecting accelerometer readings, or to steps 510 and/or 610, to continue collecting pressure and/or temperature readings.

If the step threshold requirement is not satisfied at step 830, at step 940 a combination information alert is generated. The combination information alert may provide an indication that multiple threshold requirements were not satisfied. The combination information alert may also provide the pressure readings for the specified time period, the plantar locations associated with the pressure readings, the percentage of time spent in an unacceptable pressure state for the specified period, the plantar temperature readings for the first and second times, the plantar locations associated with the plantar temperature readings, the first and second temperature differences, the rolling step count, the step count threshold, the step count baseline, methodology for generating the step count information alert, activity data for the user, and/or historical user data of the user. The combination information alert may be presented visually on an output device of the system, such as a display, or it may be provided audibly or haptically. For example, the combination information alert may be provided in the form of a pop-up notification on the display, notifying the user that their activity levels for the day are high, and their plantar pressures and/or plantar temperatures are high.

At step 950, a risk factor is generated. In this example, where the method is applied to different types of sensor readings and the information alert that is generated is a combination information alert, the generated risk factor may be a “high” risk level.

At step 960, a combination action alert may be generated. A combination action alert indicates an action that may be taken to reduce ulceration risk. If the action is taken and method 900 is repeated, at steps 530, 630, and/or 930, the threshold requirements will in theory be satisfied. The combination action alert may be presented visually on an output device of the system, such as a display, or it may be provided audibly or haptically.

For example, the combination action alert may be provided in the form of a pop-up notification on the display.

The combination action alert may include a pressure offloading alert. The pressure offloading alert may include an automated, self-directed pressure offloading procedure. The procedure may be presented on an output device of the system, and the user may follow the steps of the procedure to safely reduce pressure on their feet. By reducing pressure on their feet, inflammation may decrease to prevent the formation of ulcers.

The combination action alert may include an activity modification alert. The activity modification alert may be presented in the form of a step count goal. The step count goal may encourage users to gradually increase their activity level, to achieve the cardiovascular, metabolic, and mental health benefits of engaging in physical activity, while discouraging users from abruptly increasing activity levels in ways that might be unsafe. Alternatively, the activity modification alert may be presented in the form of a modified weight bearing activity alert. The modified weight bearing activity alert may include an automated, self-directed procedure for performing a modified weight bearing activity. The modified weight bearing activity alert may encourage users to continue to engage with physical activities, but in ways that minimize pressures on their feet to reduce inflammation and ulceration risk.

The combination action alert may include a foot examination alert. The foot examination alert may recommend that a user perform an inspection of their feet for abnormalities (e.g. visible abnormalities), abnormal colors, calluses, foot deformities, and temperature differences that can be determined by touch.

The combination action alert may include a vascular assessment requirement alert, which recommends a vascular assessment to the user. The vascular assessment requirement alert may recommend a vascular assessment including an ankle brachial index test with segmental pressures and a doppler waveform analysis.

The combination action alert may include a custom orthotic alert. The custom orthotic alert may recommend an orthotic to the user that if worn, will assist the user in offloading pressures underfoot. The custom orthotic alert may provide a recommended orthotic, where the recommended orthotic is one or more of a single dancer pad, a double dancer pad, a metatarsal bar, a metatarsal pad, a u-shaped metatarsal pad, an extrinsic post, and/or a heel lift, as well as a recommended placement for the orthotic. The recommended orthotic and the recommended placement for the orthotic may be determined based on the plantar locations associated with pressure readings determined to exceed the pressure threshold of 50 mmHg at step 520, or the first and second plantar locations associated with the first and second temperature differences that are determined to exceed the temperature threshold at step 630. In particular, if pressure readings exceed the pressure threshold, or if the first and second temperature differences exceed the temperature threshold at the second metatarsal of a user's foot, the custom orthotic alert may recommend a metatarsal pad or a u-shaped metatarsal pad to the user. If pressure readings exceed the pressure threshold, or if the first and second temperature differences exceed the temperature threshold at one or more of the metatarsal heads of a user's foot, the custom orthotic alert may recommend a single dancer pad or double dancer pads to the user.

The combination action alert may include a surgical alert. The surgical alert may recommend a surgery to the user that if performed, may prevent ulceration. The surgical alert may be generated based on the pressure readings collected for the user. In particular, an increase in the pressure readings at a user's midfoot may indicate a collapsed arch. The surgical alert may present a recommendation for a surgical relief cut in response to the increased pressures, to help relieve pain from the collapsed arch and restore function to the arch. In one example, the surgical alert recommending a relief cut is generated if pressure readings at the midfoot of a user are determined to exceed the 50 mmHg pressure threshold at step 520.

The combination action alert may include an engagement incentivization alert. The engagement incentivization alert may gamify or provide incentives for a user to increase their engagement with the input device and/or the processor/display. An example of a method that may be used to promote increased engagement of an input device is described in US Patent Application Publication No. 2023/0309933 entitled “SYSTEM AND METHOD FOR ANALYZING USE OF A WEARABLE DEVICE”, which is incorporated herein by reference.

Though FIG. 9 provides an example method of generating combination alerts that combines example method 500 (using pressure readings), example method 600 (using temperature readings), and example method 800 (using accelerometer readings), combination alerts may be generated from any combination of methods 500 to 800, or alerts generated by various other sensors. For example, combination alerts may also be generated by combining example method 500 (using pressure readings), example method 600 (using temperature readings), and example method 700 (using any sensor readings).

Referring now to FIG. 10, shown therein is an example of a risk factor hierarchy. As previously mentioned, information alerts may be generated using the sensor data collected with the input device. Collecting data using an input device with a wide variety of sensors enables different types of information alerts to be generated, which can provide a clearer understanding of risk. The risk factor hierarchy organizes the various information alerts according to likelihood of indicating an ulcer or related foot complication and provides the baseline for ordering and ranking each user in the plurality of users. In particular, when the input device 100 is the insole 101, pressure readings, temperature readings, and accelerometer readings can be collected, and pressure information alerts, temperature information alerts, adherence information alerts, step count information alerts, and combination information alerts can be generated from the sensor readings, according to methods 500 to 900. The risk factors that are generated for these information alert types may be risk factor qualitative levels, generated according to the risk factor hierarchy of FIG. 10.

According to FIG. 10, there may be four risk factor qualitative levels: a low level 1010, a medium level 1020, a medium-high level 1030, and a high level 1040. Shown at the bottom of the hierarchy is the low level 1010. Falling within the low level 1010 are pressure information alerts. Elevated pressures on the foot may be a precursor to inflammation and ulceration, but a pressure information alert on its own (i.e.

unaccompanied by other alert types) will typically indicate that inflammation and tissue damage have not yet occurred. Ulcers are preventable at this stage, if action is taken to offload the pressures on the foot in response to the alert, before inflammation can occur. Therefore, a pressure information alert is less concerning than any other alert type and is associated with the lowest level in the risk factor hierarchy.

Optionally, step count information alerts may also fall into the low level 1010. As described in method 800, a step count information alert is generated when a user's rolling step count exceeds a step count threshold, which is typically a short period of time. Abruptly increasing one's activity levels in a short period of time may create unsafe conditions for the user. However, if the step count information alert is not accompanied by another information alert type, such as a pressure information alert or a temperature information alert (i.e. the increase in activity does not have repercussions on the user's plantar pressures and temperatures), the increase in activity may not be concerningly unsafe for the user. Increased activity levels may be a precursor to inflammation and ulceration, but a step count information alert on its own (i.e. unaccompanied by other alert types) will typically indicate that inflammation and tissue damage have not yet occurred and ulceration is still preventable. Therefore, step count information alerts, unaccompanied by other alert types, are associated with the low level 1010 in the risk factor hierarchy.

The medium level 1020 is shown above the low level 1010 on the risk factor hierarchy. Temperature information alerts fall into the medium level 1020. Temperature information alerts have a higher risk factor than pressure information alerts and step count information alerts, as temperature information alerts indicate that inflammation and tissue damage may be present on the foot, and ulceration is progressed to a further stage than if only a pressure information alert or a step count information alert is generated. Therefore, a temperature information alert would be considered indicative of a higher ulceration risk for the user, and a higher risk factor would be generated for the temperature information alert than the pressure information alert.

The medium-high level 1030 is shown above the medium level 1020 on the hierarchy. Falling within the medium-high level 1030 are adherence information alerts. Adherence information alerts are higher on the risk factor hierarchy than pressure and temperature information alerts, because if a user fails to wear the insoles for the required usage time, it will not be possible for the system to determine the condition of the user's feet and the progression of ulcers on the user's feet. If the condition of the user's feet is unknown, progression of ulcers is unpreventable. In particular, if the user does not wear the device, pressure information alerts and temperature information alerts cannot be generated, to instruct the user to offload pressures on their feet. Therefore, an unknown condition poses a higher risk than a known condition, where actionable steps can be taken to reduce ulceration risk.

Lastly, the high level 1040 is shown at the top of the hierarchy. Combination information alert falls into the high level 1040. As previously mentioned, combination information alerts are generated when more than one threshold requirement is not met (i.e. when two or more information alert types would have otherwise been generated). Combination information alerts are highest on the hierarchy because a combination of potential issues may indicate further progression of ulceration than just a single alert type, and may indicate progressing ulceration at multiple plantar locations.

The risk factor hierarchy additionally conveys the relative number of information alerts that are expected to be generated for each information alert type. Pressure information alerts are expected to be generated the most, since these are associated with the lowest level of risk and are associated with pre-ulceration. Combination information alerts are expected to be generated the least, since they cover the highest risk and indicate the furthest progression of ulceration compared to the other levels.

FIG. 10 shows one example of risk factor hierarchy for the input device 100. Other risk factor hierarchies may be used. For example, in a different example, the combination information alerts fall into the medium-high level, and the adherence information alerts fall into the high level. Other risk factor hierarchies can also be developed for different input devices, which collect different types of sensor readings and produce different information alert types.

While the above description provides examples of one or more methods, systems, or devices, it will be appreciated that other methods, systems, or devices may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for generating a risk score, comprising:
for each user in a plurality of users, collecting a plurality of sensor readings over a time period using an input device worn by the user, the input device configured to collect the plurality of sensor readings;
calculating one or more comparison metrics based on the plurality of sensor readings;
determining whether the one or more comparison metrics satisfies one or more threshold requirements;
generating a plurality of information alerts if the one or more comparison metrics does not satisfy the one or more threshold requirements in the time period, each information alert corresponding to an information alert type;
generating a risk factor for each information alert based on the information alert type;
generating one or more alert totals for the time period based on the plurality of information alerts;
generating a risk score for the user based on each of the risk factors and the one or more alert totals; and
ordering the plurality of users in a list according to the risk score for each user.

2. The method of claim 1, wherein ordering the plurality of users in the list comprises arranging the plurality of users in descending order according to the risk score for each user.

3. The method of claim 1, further comprising generating a care timeline for each user based on the risk score, wherein high risk scores are associated with urgent care timelines.

4. The method of claim 1, wherein the information alert type is one of a pressure information alert, a temperature information alert, an adherence information alert, and a combination information alert.

5. The method of claim 1, wherein generating the one or more alert totals for the time period comprises counting a total number of information alerts in the time period or counting a total number of information alerts corresponding to each information alert type in the time period.

6. The method of claim 1, wherein generating the risk score further comprises using additional data, wherein the additional data comprises foot examination data, sensor alert patterns, scan data for the foot of the user, historical user data, and the historical user data comprises historical ulceration data, historical foot data, historical amputation data, historical foot surgery data, historical social data, nutritional status, historical gait data, historical mobility data, historical medication user, historical comorbidity data, and/or historical lab test data.

7. The method of claim 6, further comprising calculating a tie breaking score when two users have the same risk score, wherein the tie breaking score is calculated using the one or more alert totals and/or the additional data.

8. The method of claim 1, wherein the plurality of sensor readings comprises a plurality of pressure readings, the one or more comparison metrics comprises a percentage of time spent in an unacceptable pressure state based on the plurality of pressure readings, the threshold requirement comprises a percentage threshold requirement, the percentage threshold requirement is not satisfied when the percentage of time spent in the unacceptable pressure state exceeds a percentage threshold, and the plurality of information alerts comprises a pressure information alert.

9. The method of claim 1, wherein the plurality of sensor readings comprises a plurality of temperature readings, the one or more comparison metrics comprises a first temperature difference and a second temperature difference, the threshold requirement comprises a temperature threshold requirement, the temperature threshold requirement is not satisfied when the first temperature difference and the second temperature difference exceed a temperature threshold, and the plurality of information alerts comprises a temperature information alert.

10. The method of claim 1, wherein the one or more comparison metrics comprises a usage time, the threshold requirement comprises a usage threshold requirement, the usage threshold requirement is not satisfied when the usage time is below a usage threshold, and the plurality of information alerts comprises an adherence information alert.

11. The method of claim 1, wherein the plurality of sensor readings comprises a plurality of accelerometer readings, the one or more comparison metrics comprises a rolling step count, the threshold requirement comprises a step threshold requirement, the step threshold requirement is not satisfied when the rolling step count exceeds a step count threshold, and the plurality of information alerts comprises a step count information alert.

12. The method of claim 1, wherein the plurality of sensor readings comprises a plurality of accelerometer readings and a plurality of pressure readings, the one or more comparison metrics comprises a rolling step count and a percentage of time spent in an unacceptable pressure state based on the plurality of pressure readings, the threshold requirement comprises a step threshold requirement and a percentage threshold requirement, the step threshold requirement is not satisfied when the rolling step count exceeds a step count threshold and the percentage threshold requirement is not satisfied when a percentage of time spent in an unacceptable pressure state exceeds a percentage threshold, and the plurality of information alerts comprises a combination information alert.

13. The method of claim 1, wherein the plurality of sensor readings comprises a plurality of accelerometer readings and a plurality of temperature readings, the one or more comparison metrics comprises a rolling step count, a first temperature difference, and a second temperature difference, the threshold requirement comprises a step threshold requirement and a temperature threshold requirement, the step threshold requirement is not satisfied when the rolling step count exceeds a step count threshold and the temperature threshold requirement is not satisfied when the first temperature difference and the second temperature difference exceed a temperature threshold, and the plurality of information alerts comprises a combination information alert.

14. The method of claim 1, wherein the input device is footwear, an insole, or a pair of insoles.

15. A system for generating a risk score, comprising:
an input device worn by each user in a plurality of users, the input device including:
a plurality of sensors, the plurality of sensors configured to collect a plurality of sensor readings over a time period;
a processor in communication with the input device, the processor configured to:
receive the plurality of sensor readings from the input device;
calculate one or more comparison metrics based on the plurality of sensor readings;
determine whether the one or more comparison metrics satisfies one or more threshold requirements;
generate a plurality of information alerts if the one or more comparison metrics does not satisfy the one or more threshold requirements in the time period, each information alert corresponding to an information alert type;
generate a risk factor for each information alert based on the information alert type;
generate one or more alert totals for the time period based on the plurality of information alerts;
generate a risk score for the user based on each of the risk factors and the one or more alert totals; and
order the plurality of users in a list according to the risk score for each user.

16. The system of claim 15, wherein the processor is configured to order the plurality of users in the list in descending order according to the risk score for each user.

17. The system of claim 15, wherein the processor is further configured to generate a care timeline for each user based on the risk score, wherein high risk scores are associated with urgent care timelines.

18. The system of claim 15, wherein the information alert type is one of a pressure information alert, a temperature information alert, an adherence information alert, and a combination information alert.

19. The system of claim 15, wherein the processor is further configured to generate the risk score using additional data, wherein the additional data comprises foot examination data, sensor alert patterns, scan data for the foot of the user, historical user data, and the historical user data comprises historical ulceration data, historical foot data, historical amputation data, historical foot surgery data, historical social data, nutritional status, historical gait data, historical mobility data, historical medication user, historical comorbidity data, and/or historical lab test data.

20. The system of claim 15, wherein the input device is footwear, an insole, or a pair of insoles.
